(19) United States
(12) Patent Application Publication
(10) Pub. No.: US 2024/0115594 A1
(43) Pub. Date: Apr. 11, 2024

(54) DINUCLEOTIDES AND THEIR USE IN TREATING CANCER

(71) Applicant: MAIA Biotechnology, Inc., Chicago, IL (US)

(72) Inventors: Sergei Gryaznov, San Mateo, CA (US); Jerry Shay, Dallas, TX (US)

(73) Assignee: MAIA Biotechnology, Inc., Chicago, IL (US)

(21) Appl. No.: 18/352,220

(22) Filed: Jul. 13, 2023

Related U.S. Application Data
(60) Provisional application No. 63/388,688, filed on Jul. 13, 2022.

(51) Int. Cl. A61K 31/7084; A61P 35/00

(52) U.S. Cl. CPC A61K 31/7084; A61P 35/00

(57) ABSTRACT

Disclosed herein are dinucleotide compounds that target telomers in cancer cells and methods for using the dinucleotide compounds to treat cancers alone or before administration of checkpoint inhibitors.

### PRIORITY CLAIM

This Application claims the benefit of priority to U.S. Provisional Application Ser. No. 63/388,688, entitled Dinucleotides and Their Use in Treating Cancer, filed 13 Jul. 2022.

### FIELD OF THE DISCLOSURE

The present invention is in the field of cancer treatments. In particular cancer treatments using dinucleotide compounds that target telomeres in cancer cells.

### BACKGROUND OF THE DISCLOSURE

Telomeres are found at both ends of eucaryotic chromosomes. These DNA-protein structures protect the genome from nucleolytic degradation, unneeded recombination, intrachromosomal fusion and repair (Shammas, M., (2011), Curr Opin, Clin. Nutr Metab Care, 14(1):28-34). Telomere length shortens with each cell division due to the end replication problem and the absence of a telomere maintenance mechanism (Greider C. W, (1996), Annu Rev Biochem. 65:337-65). However, unicellular eukaryotes, germline cells and immortal cancer cells maintain their telomeres at a constant length almost always by activating the enzyme telomerase (Greider and Blackburn, (1985) Cell,

December; 43(2 Pt 1):405-13; McEachern and Blackburn, (1996), Genes & Dev. 1996; 10:1822-1834; Morin G B., (1989), Cell, 59:521-529; Nakamura et al., (1997), Science, 15:277(5328); Singer and Gottschling, (1994) Science, 266:404-409; Yu et al., (1990), Nature, 344:126-132). Telomerase is a reverse transcriptase enzyme that elongates telomeres by adding TTAGGG repeats to the ends of chromosomes and is expressed in about 90% of human tumors, but not in most normal cells (Jafri, M. A., et al. Genome Medicine, 2016, 8:69). Thus, telomerase is an attractive target to develop anti-cancer therapies. Most therapies targeting telomerase have focused on inhibiting telomerase (Andrews L G, Tollefsbol T O. Mol Biol. 2007; 405:1-7); however, such inhibitors have not fared well in clinical studies (Zhang G., Shay W S. Oncotarget, 2018, vol 9(88):35803-35804).

Other cancer drugs are being developed which instead of inhibiting telomerase use telomerase for incorporation into telomeres resulting in DNA damage, senescence or crisis in telomerase positive cancer cells. One such compound is the nucleoside analogue, 6-thio-2′-deoxyguanosine (6-thio-dG). Its incorporation into de novo synthesized telomeres by telomerase is known to induce damage on telomeric DNA (Mender et al., (2015), Cancer Discov., 5(1):82-95; Mender et al., (2015), Oncoscience, 2(8):693-695). This results in rapid tumor shrinkage or growth arrest in many tumor-derived xenograft models with minimal side effects (Mender et al., (2018), Neoplasia, 20(8):826-837); Sengupta et al., (2018), Mol Cancer Ther., July; (17(7):1504-1514). The most important advantage of this telomere-targeted therapy over direct telomerase inhibitors is that 6-thio-dG does not have a long lag period for tumor killing effects. Additionally, it does not directly inhibit telomerase but is preferentially recognized by telomerase over other polymerases and incorporated into the telomeres resulting in an immediate DNA chain termination. Importantly, its effect is independent of initial telomere length by hijacking tumor telomerase to make unstable telomeres (Mender et al., (2015) Oncoscience, 2(8):693-695).

Disclosed below are new telomere-targeting compounds useful for treating cancer.

### SUMMARY OF THE EMBODIMENTS

Disclosed herein are dinucleotide compounds or a pharmaceutically acceptable salt thereof that are telomere targeting compounds useful for treating cancers. In some embodiments, the dinucleotide compounds are telomerase-mediated telomere targeting compounds useful for treating cancers

In some embodiments the dinucleotide compounds disclosed herein have the structure of Formula I

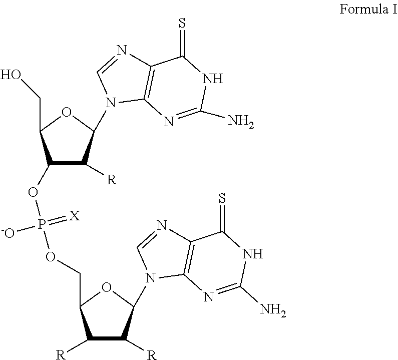

wherein R is independently H or OH and X is O or S. In some embodiments X is O. In other embodiments X is S.

When X is O, the dinucleotide compounds of Formula I can have the following structures:

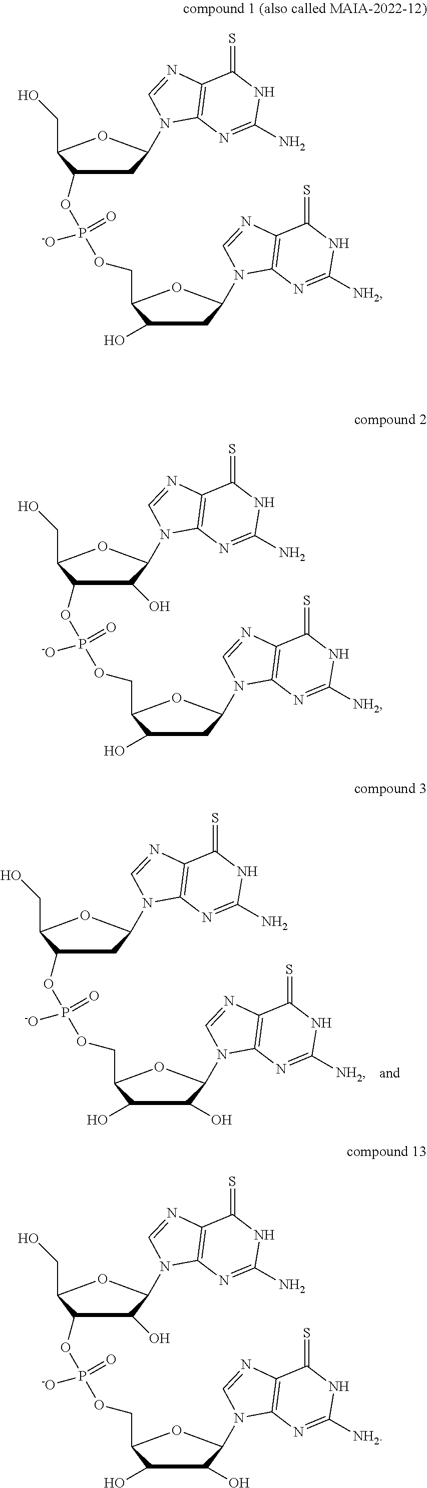

When X is S the dinucleotide compounds of Formula I can have the following structures:

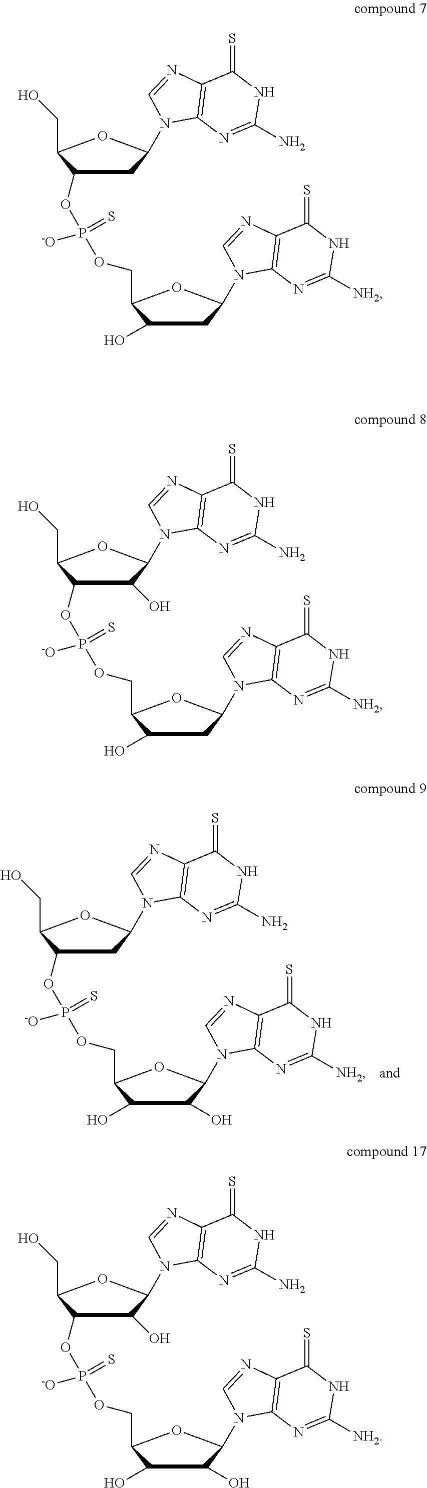

In other embodiments the dinucleotide compounds can have one or more of the following structures:

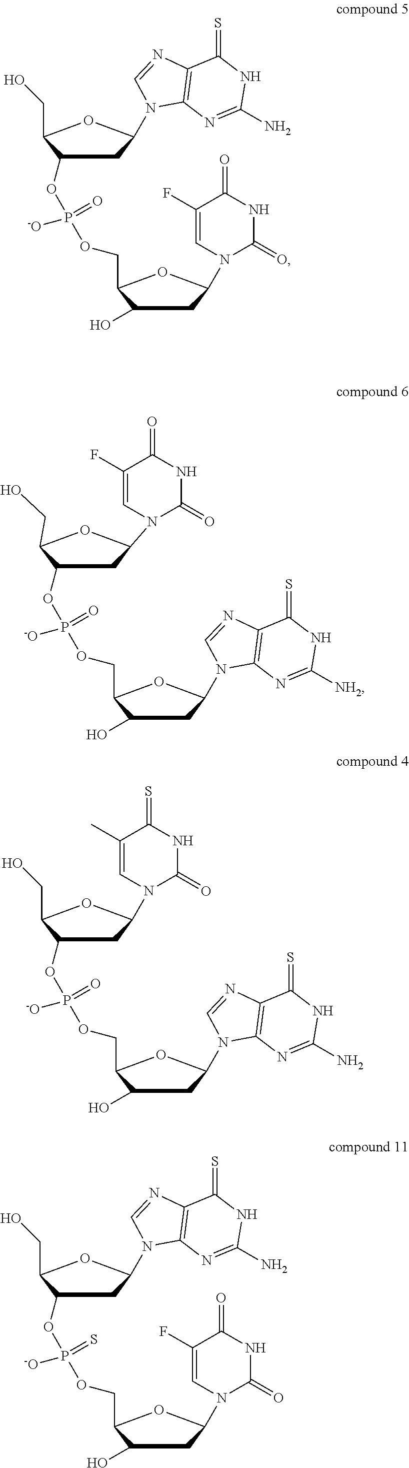

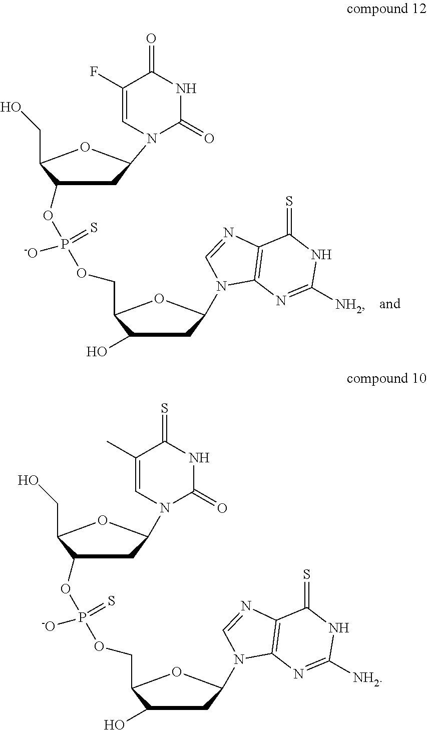

Also disclosed herein are enantiomers of compound 11 that have the following stereochemistry:

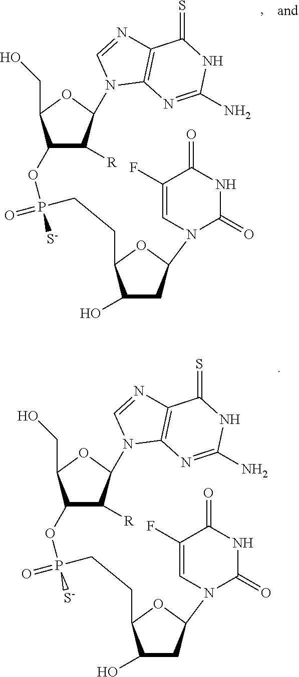

In another embodiment are enantiomers of compound 12 that have the following stereochemistry:

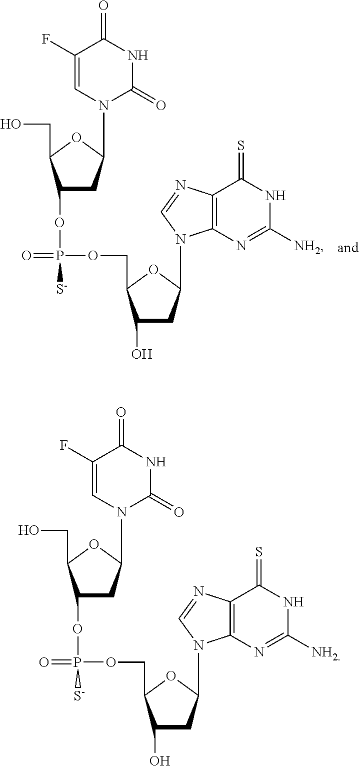 [SMILES: Nc1nc2c(ncn2[C@H]2C[C@@H](O)[C@@H](CO[P@@](=O)([S-])O[C@@H]3C[C@H](n4cc(F)c(=O)[nH]c4=O)O[C@@H]3CO)O2)c(=S)[nH]1 ; Nc1nc2c(ncn2[C@H]2C[C@@H](O)[C@@H](CO[P@@](=O)([S-])O[C@@H]3C[C@H](n4cc(F)c(=O)[nH]c4=O)O[C@@H]3CO)O2)c(=S)[nH]1]

In some aspects, the dinucleotide compounds have the structure of Formula II:

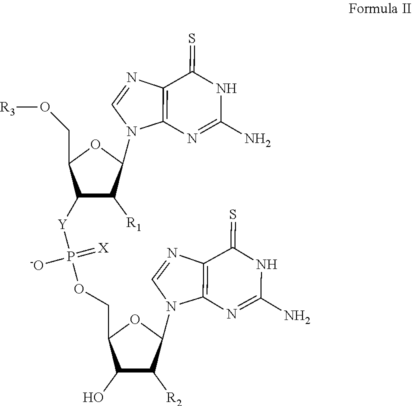

Where R₁ and R₂ are independently H or OH;
X is O or S;
Y is O; and
R₃ is selected from the group consisting of a phosphate group, thio-phosphate group, palmitic acid, tocopherol, and cholesterol group.

In some embodiments of Formula II, R₁ and R₂ are independently H or OH, X is O, Y is O, and R₃ is a cholesterol group. In some embodiments, the dinucleotide compounds of Formula II have the following structures,

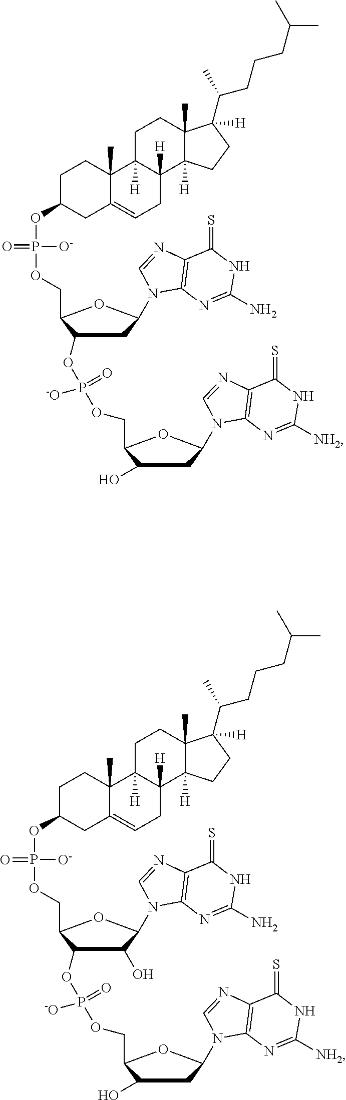

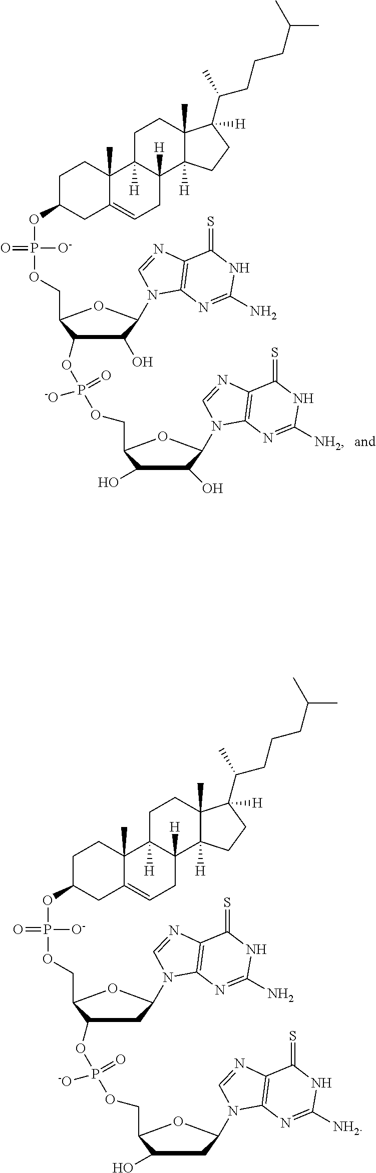

In some embodiments of Formula II, R₁ and R₂ are independently H or OH, X and Y are O, and R₃ is a phosphate group. In some embodiments, the dinucleotide compounds of Formula II have the following structures:

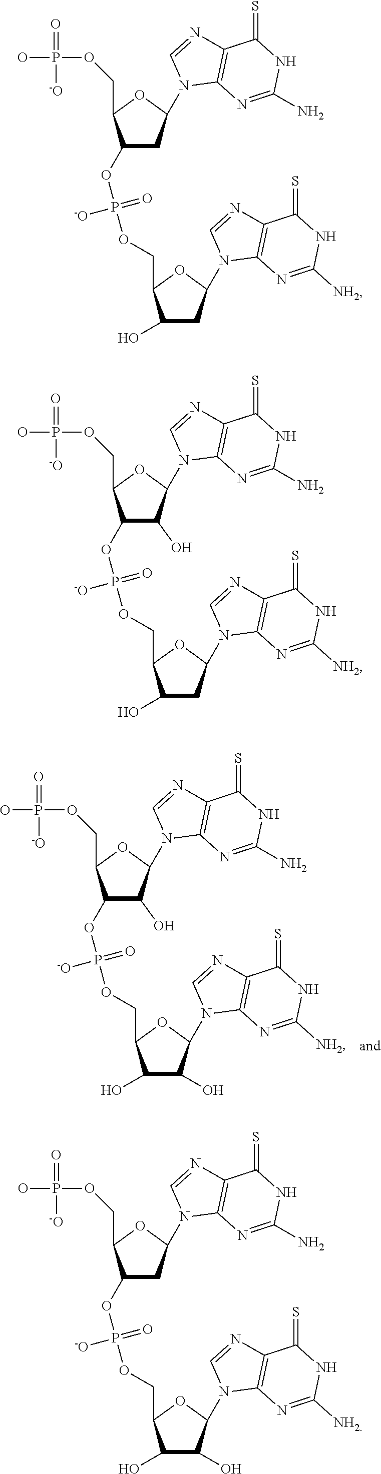

In other embodiments of Formula II, R₁ and R₂ are independently H or OH, X and Y are independently O, and R₃ is a thio-phosphate group. In some embodiments, the dinucleotide compounds of Formula II have the following structures:

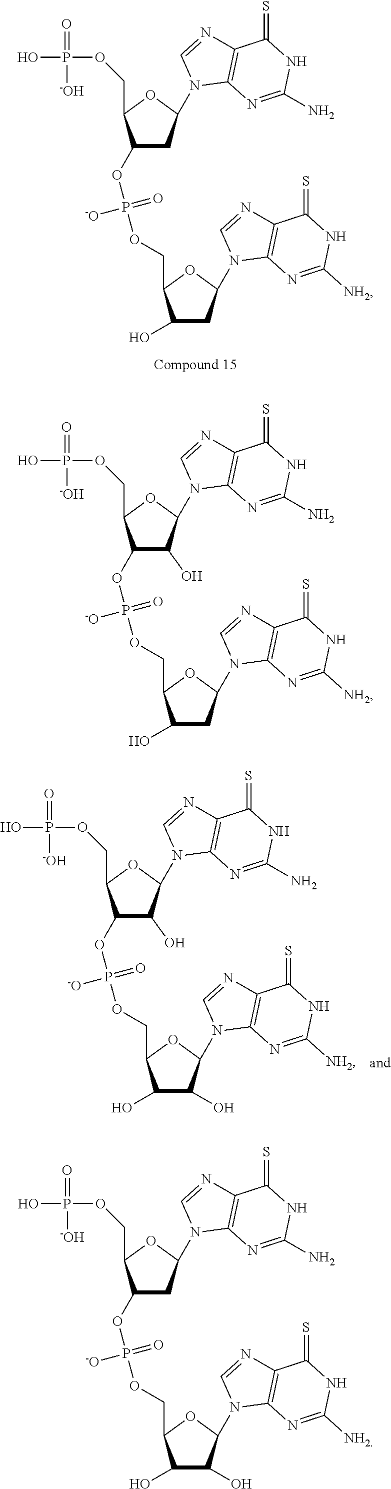

In yet other embodiments of Formula II, R₁ and R₂ are independently H or OH; X and Y are independently O, and R₃ is palmitic acid. In some embodiments, the dinucleotide compounds of Formula II have the following structures:

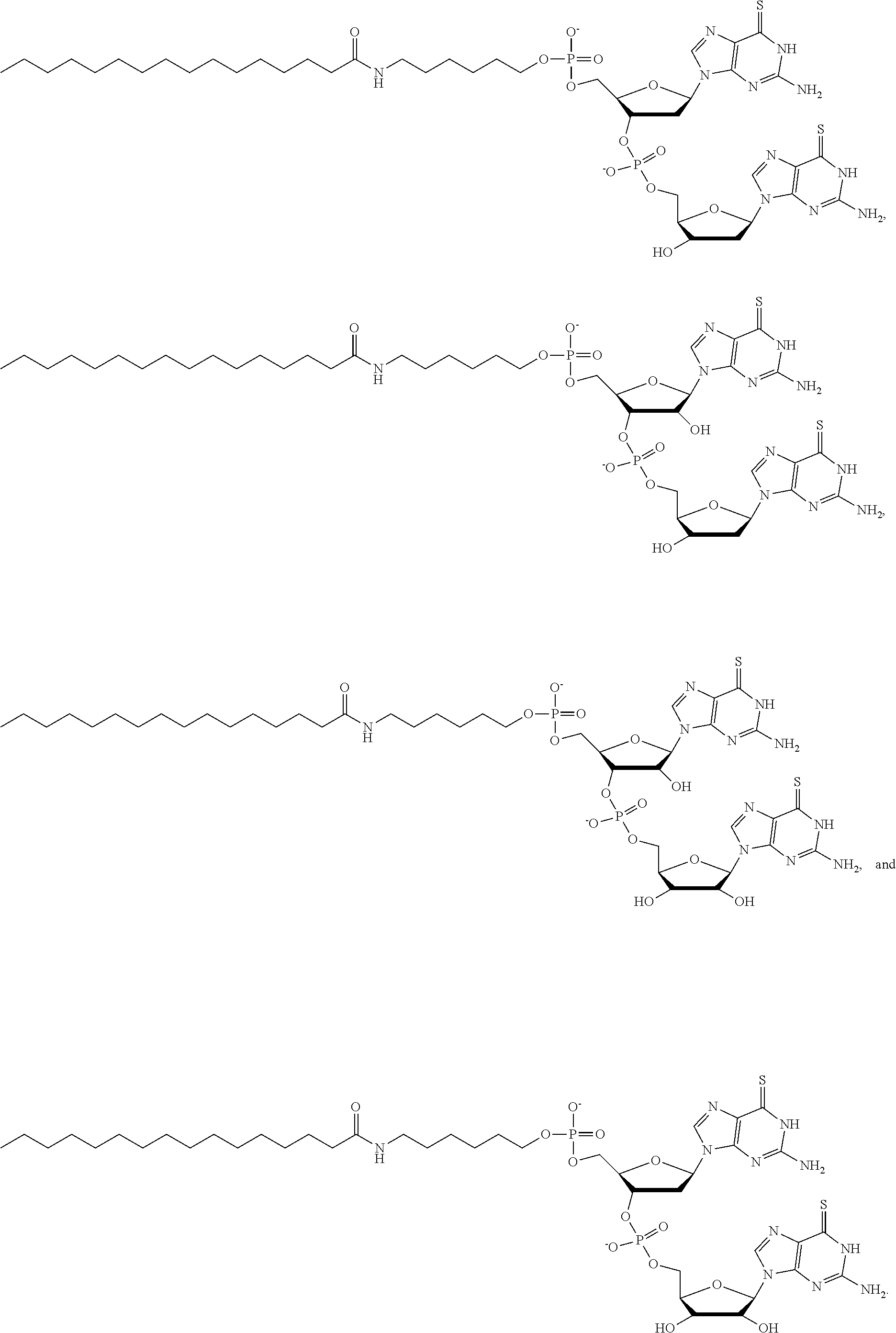

In still other embodiments of Formula II, R₁ and R₂ are independently H or OH, X and Y are independently O, and R₃ is tocopherol. In some embodiments, the dinucleotide compounds of Formula II have the following structures:

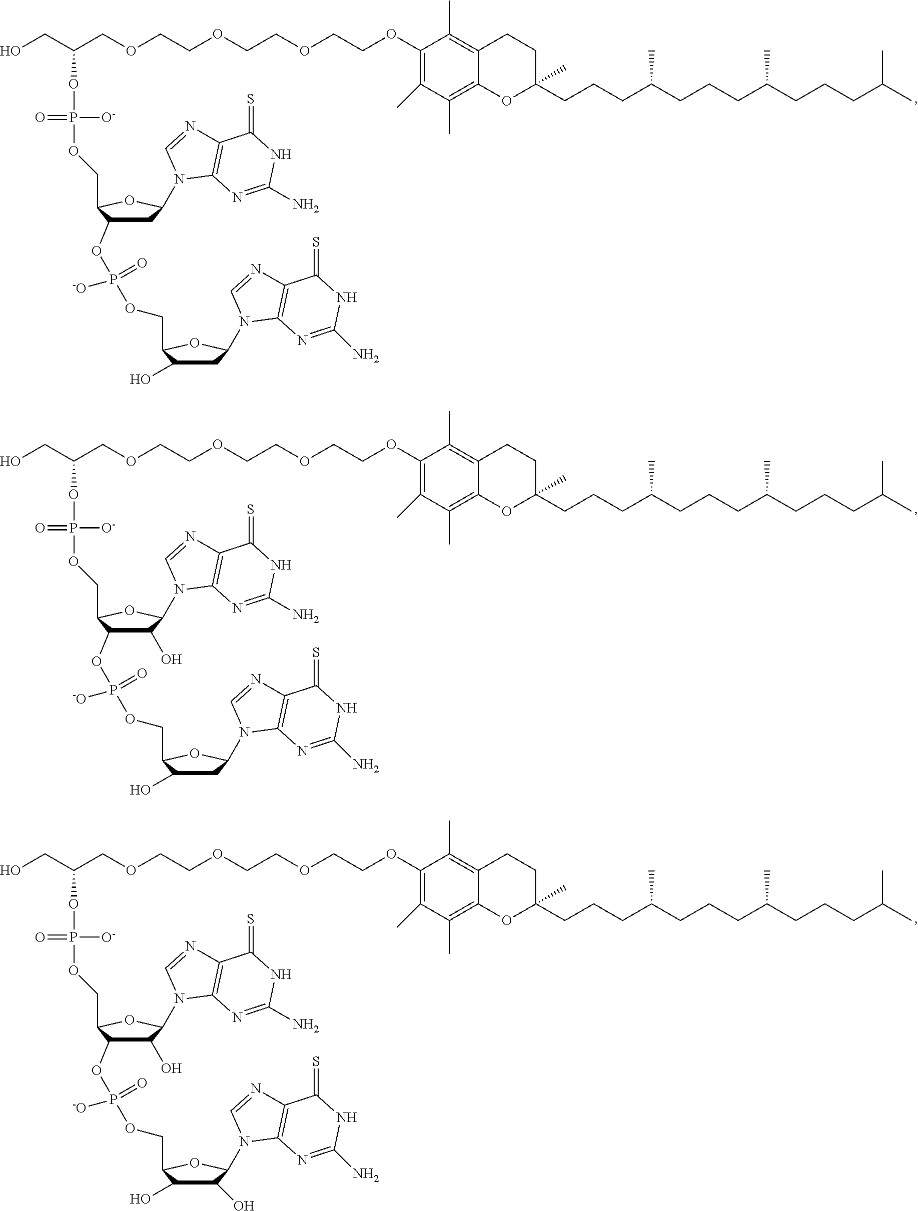

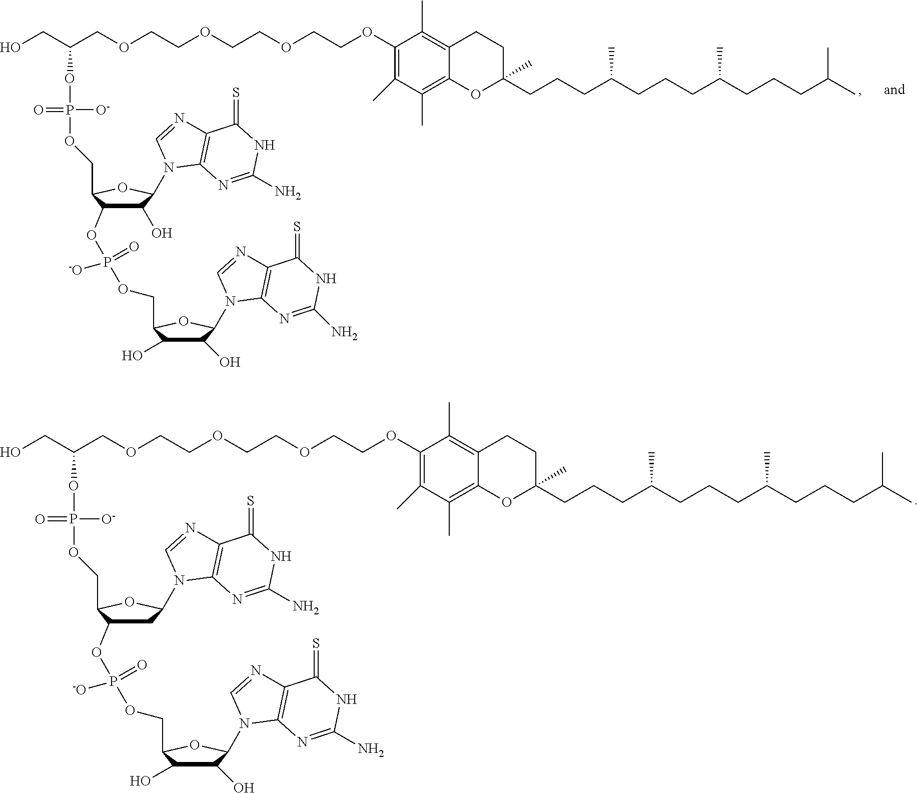

In other embodiments of Formula II, R₁ and R₂ are independently H or OH, X is S, Y is O, and R₃ is a cholesterol group. In some embodiments, the dinucleotide compounds of Formula II have the following structures:

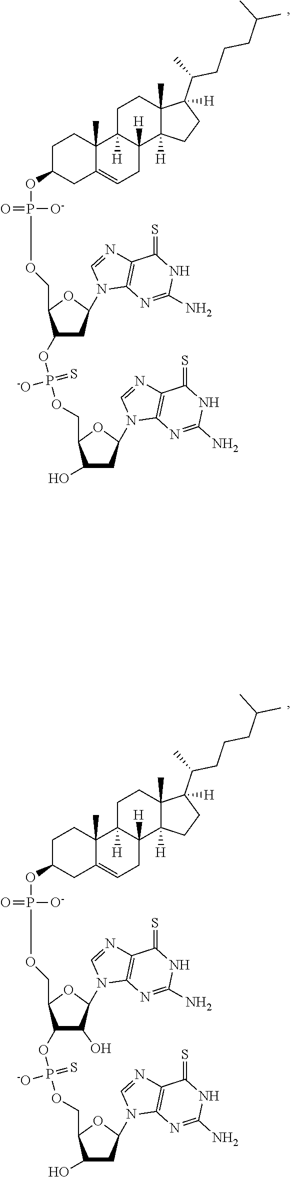

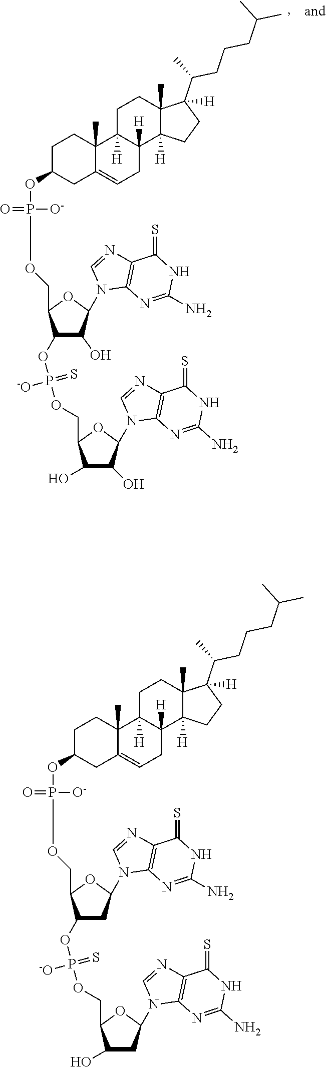

In other embodiments of Formula II, R₁ and R₂ are independently H or OH, X is S, Y is O, and R₃ is a phosphate group. In some embodiments, the dinucleotide compounds of Formula II have the following structures:

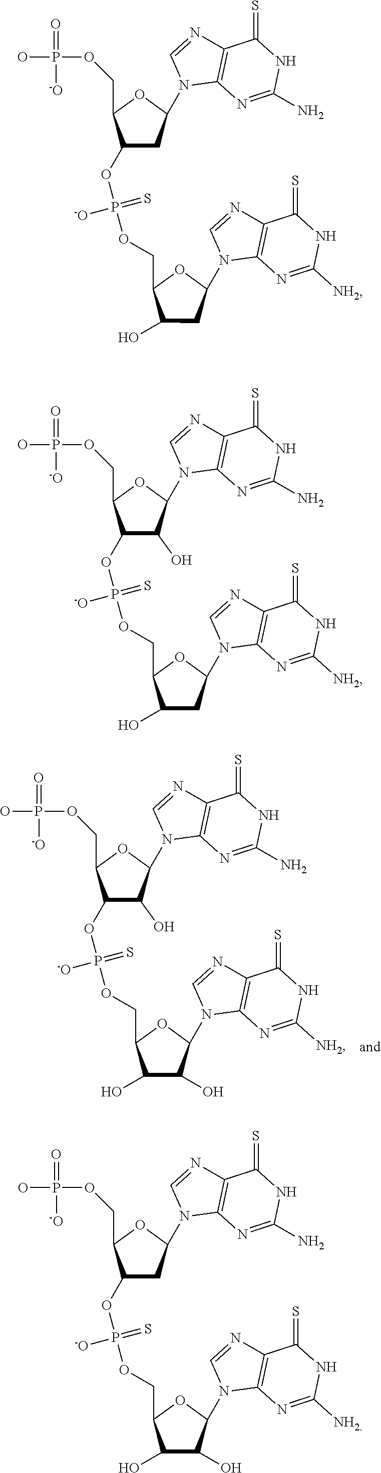

In some embodiments of Formula II, R₁ and R₂ are independently H or OH, X is S, Y is O, and R₃ is a thio-phosphate group. In some embodiments, the dinucleotide compounds of Formula II have the following structures:

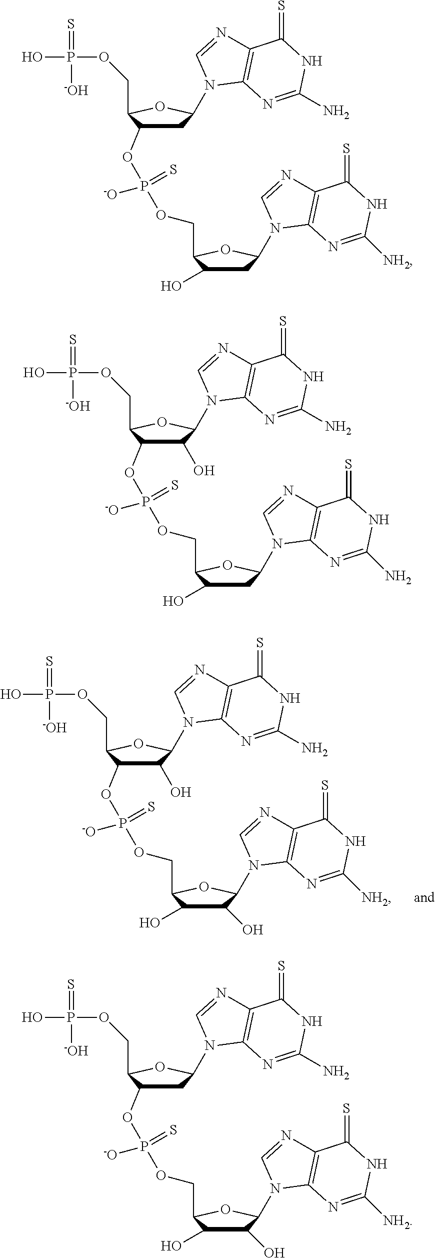

In some embodiments of Formula II, R₁ and R₂ are independently H or OH, X is S, Y is O, and R₃ is palmitic acid. In some embodiments, the dinucleotide compounds of Formula II have the following structures:

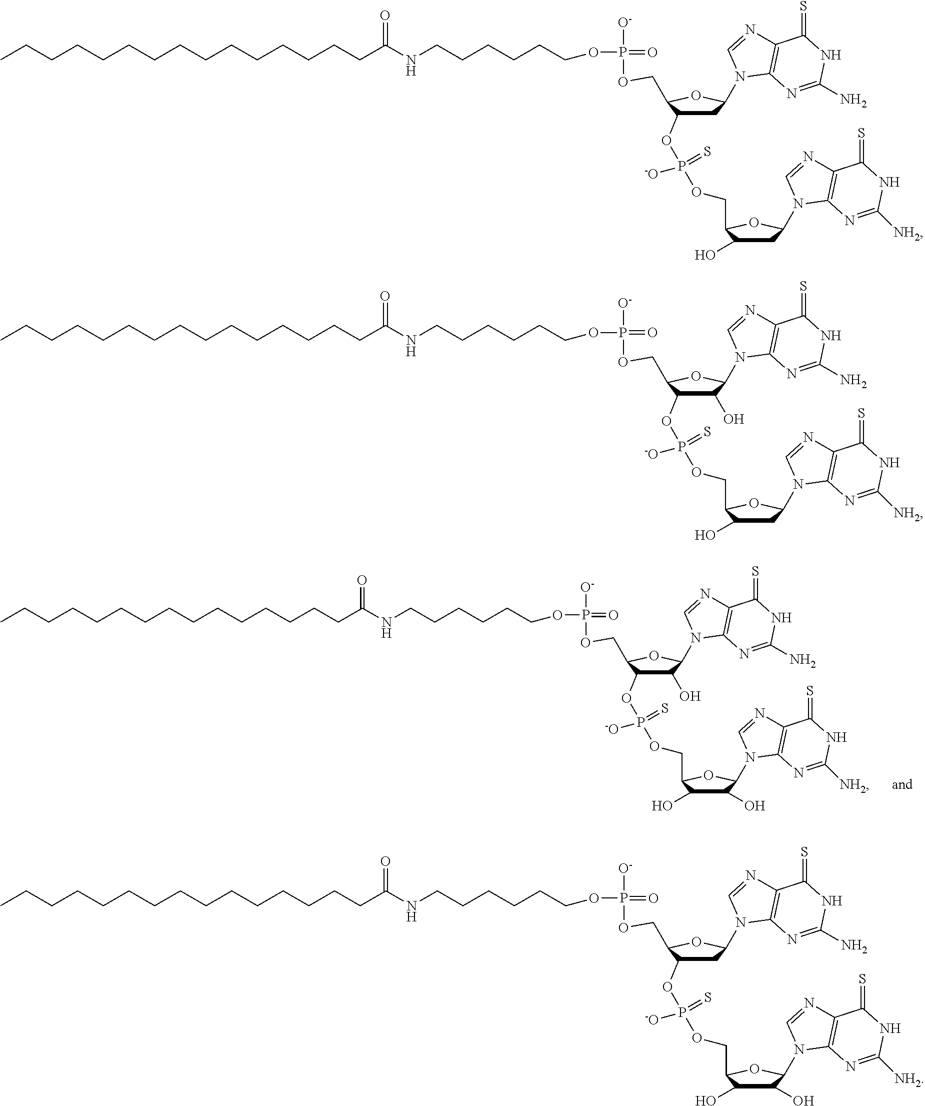

In other embodiments of Formula II, R₁ and R₂ are independently H or OH, X is S, Y is O, and R₃ is tocopherol. In some embodiments, the dinucleotide compounds of Formula II have the following structures:

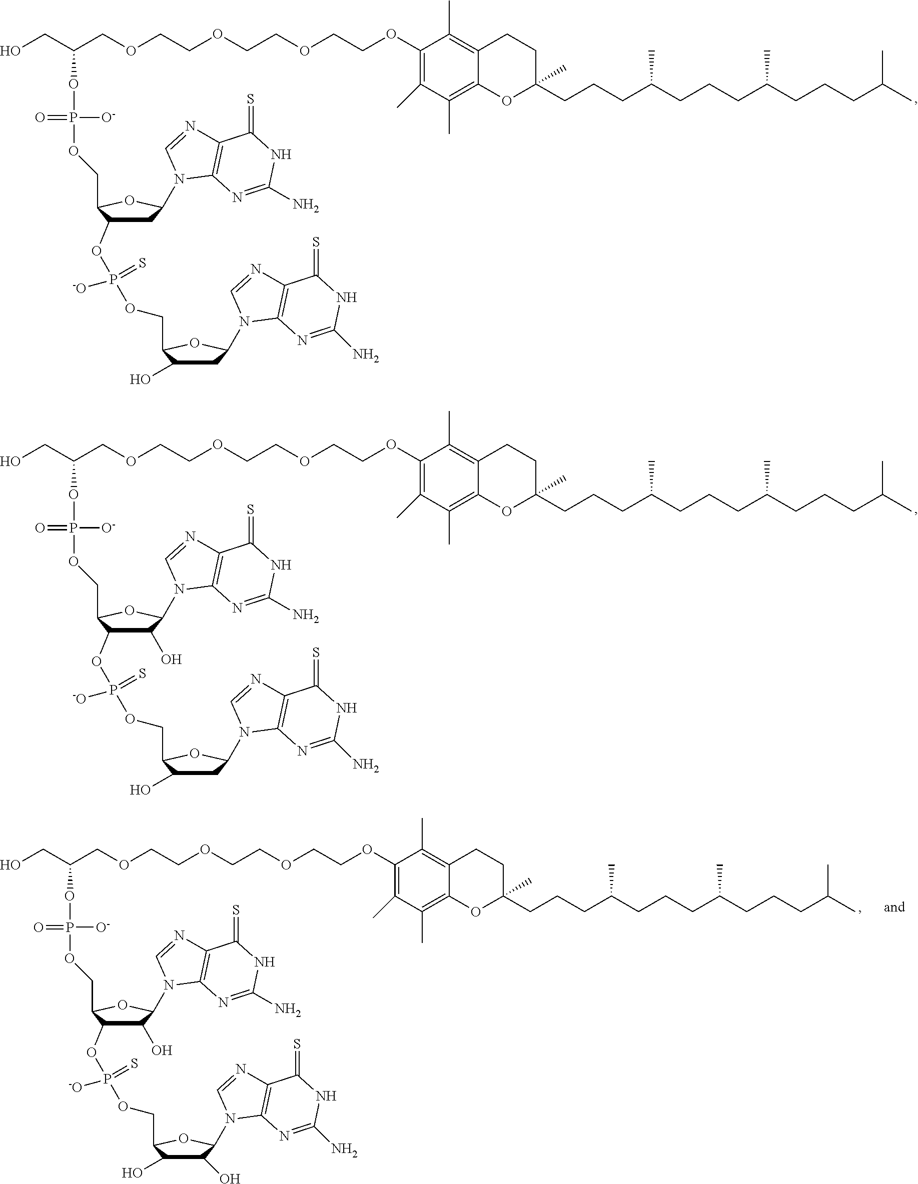

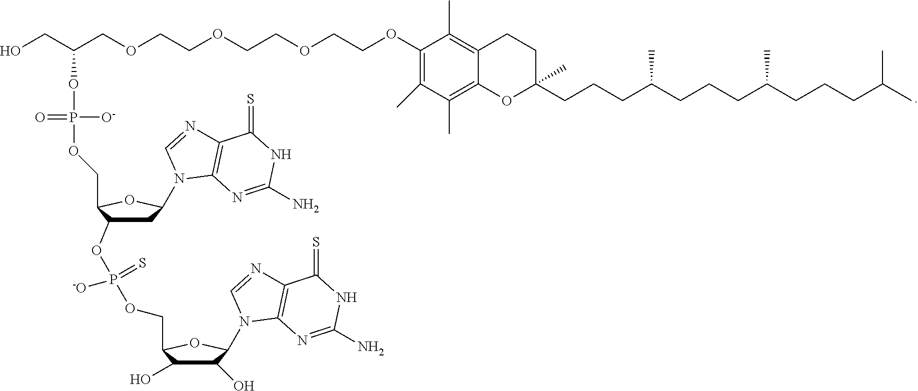

In other embodiments are dinucleotide compounds comprising the structure of Formula III:

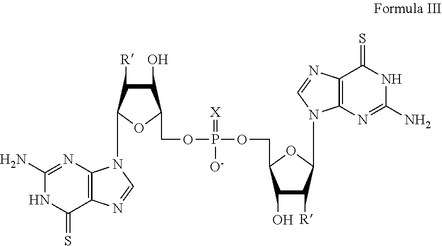

wherein X is O or S and R′ is independently H or OH. In some embodiments, the dinucleotide compounds of Formula III have the following structures:

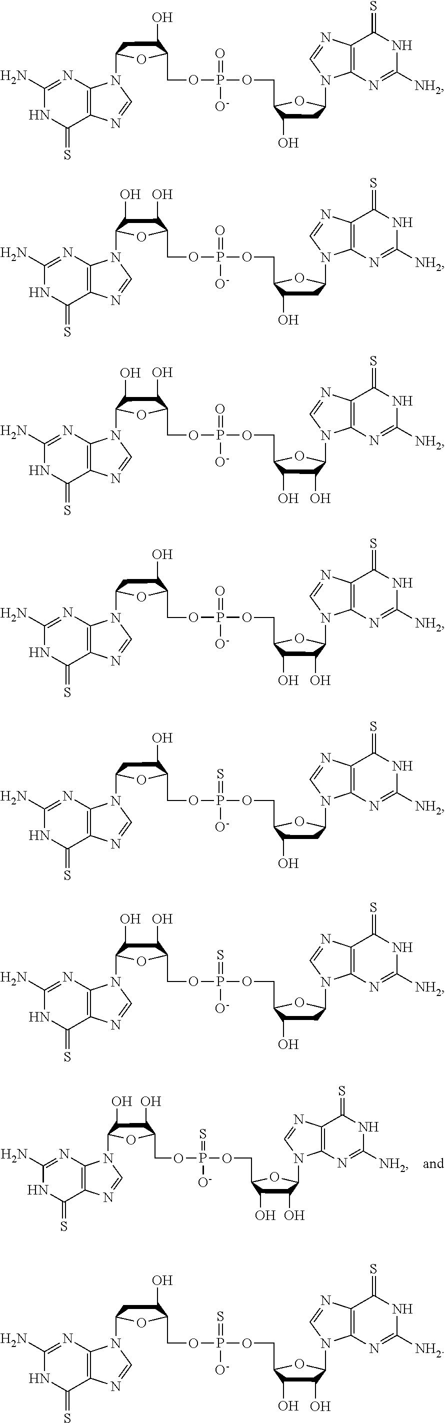

In other embodiments are dinucleotide compounds comprising the structure of Formula IV.

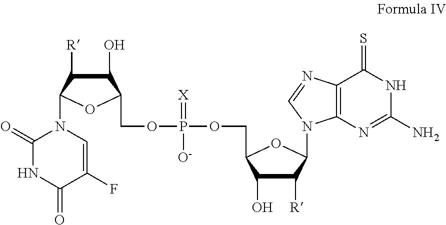

wherein X is O or S and R′ is independently H or OH.

In some embodiments, the dinucleotide compounds of Formula IV have the following structures:

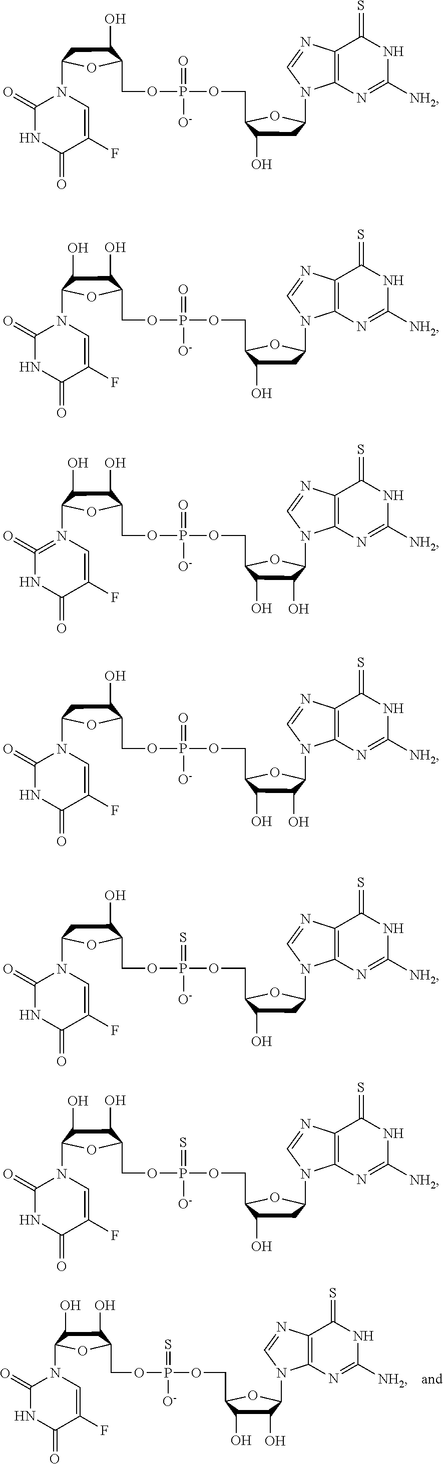

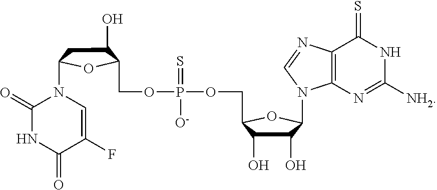

In other embodiments are dinucleotide compounds comprising the structure of Formula V wherein R′ is independently H or OH.

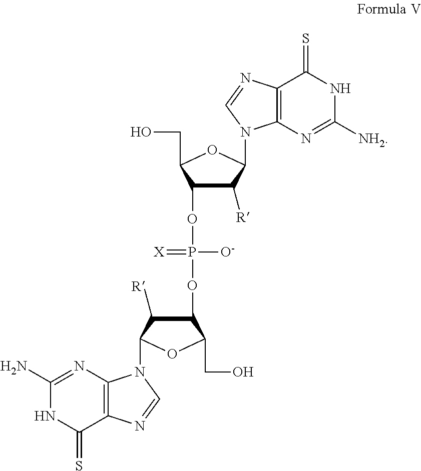

In some embodiments, the dinucleotide compounds of Formula V have the following structures:

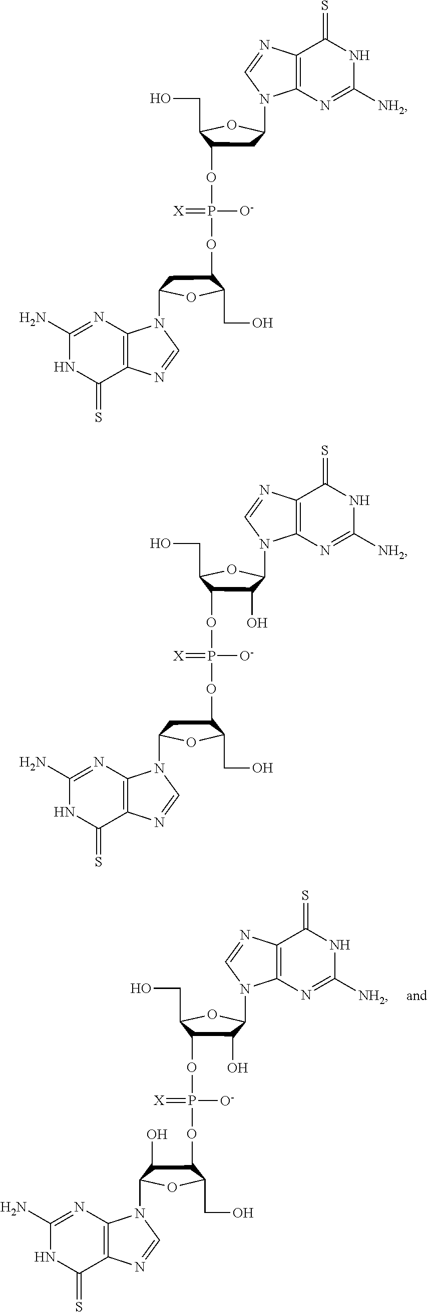

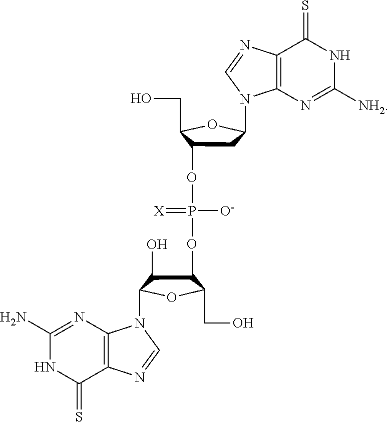

In yet other embodiments are dinucleotide compounds comprising the structure of Formula VI wherein R′ is independently H or OH

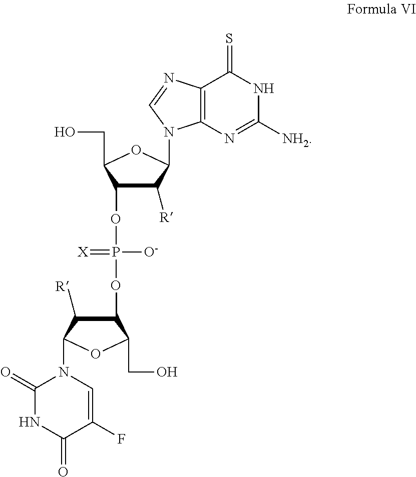

In some embodiments, the dinucleotide compounds of Formula VI have the following structures:

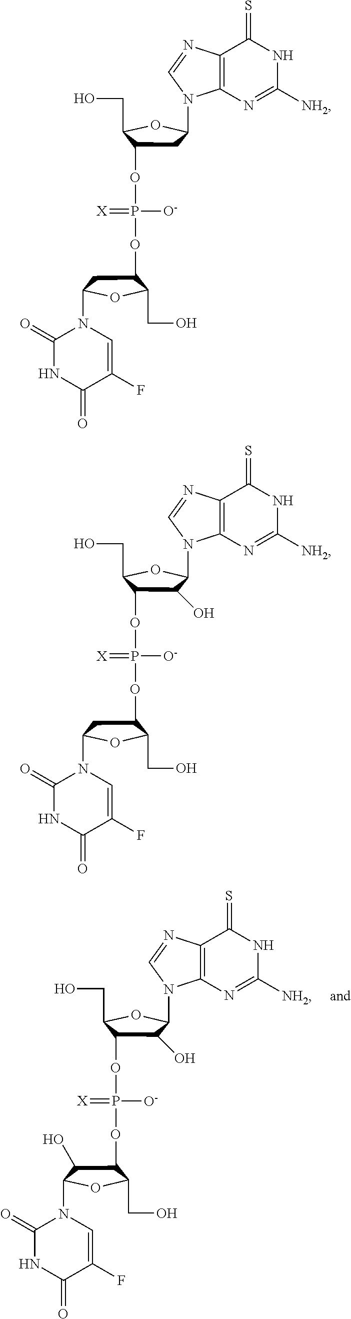

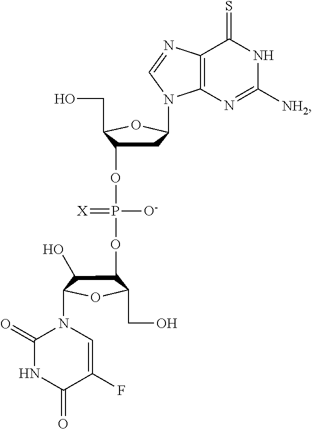

In other embodiments are dinucleotide compounds comprising the structure of Formula VII wherein R′ is independently H or OH and X is S or O.

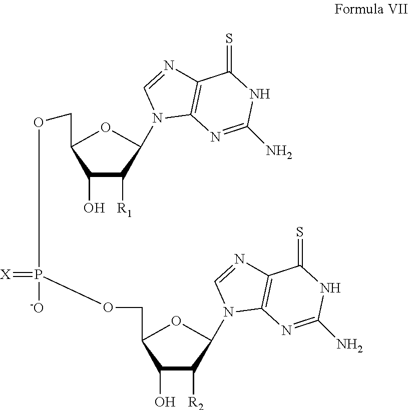

In some embodiments, the dinucleotide compounds of Formula VII have the following structures wherein X is O and R₁ and R₂ are H or OH:

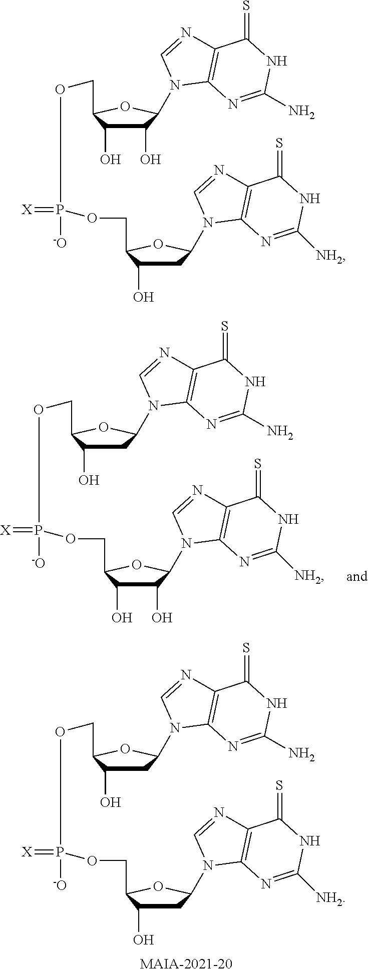

In some embodiments, the dinucleotide compounds of Formula VII have the following structures wherein X is S and R₁ and R₂ are H or OH:

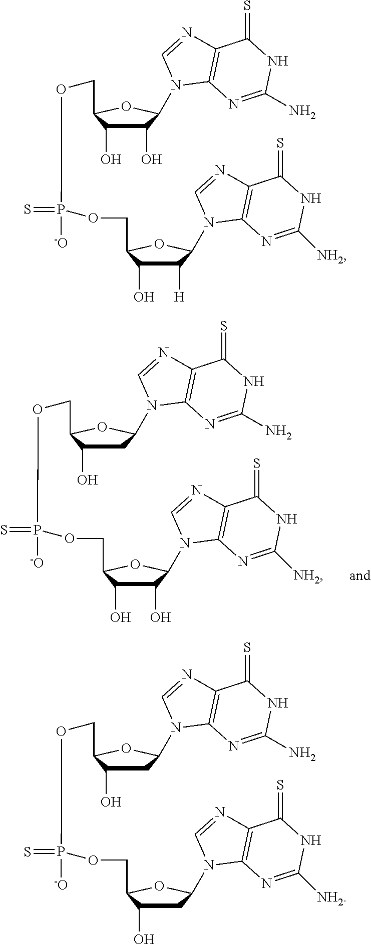

Another aspect of the disclosure are pharmaceutical salts of the dinucleotide compounds disclosed herein.

In yet other embodiments, disclosed herein are pharmaceutical compositions comprising a dinucleotide compound as described herein. In some embodiments a pharmaceutical formulation can include a least one pharmaceutically acceptable excipient.

Another embodiment disclosed herein is a process for preparing the pharmaceutical formulations comprising combining the dinucleotides compounds discussed herein with at least one pharmaceutically acceptable excipient.

In still other embodiments, disclosed herein are methods of treating a subject who has cancer, comprising administrating to the subject one or more of the dinucleotide compounds discussed herein, wherein the cancer is selected from one or more of breast cancer, prostate cancer, colon cancer, stomach cancer, esophagus, liver, squamous cell cancer, small-cell lung cancer, non-small cell lung cancer, ovarian cancer, cervical cancer, gastrointestinal cancer, pancreatic cancer, glioblastoma, liver cancer, biliary tract, bladder cancer, hepatoma, colorectal cancer, rectal cancer, uterine cancer, cervical cancer, endometrial carcinoma, salivary gland carcinoma, mesothelioma, kidney cancer, vulval cancer, pancreatic cancer, thyroid cancer, hepatic carcinoma, testicular cancer, skin cancer, melanoma, brain cancer, neuroblastoma, myeloma, various types of head and neck cancer, acute lymphoblastic leukemia, acute myeloid leukemia, Merkel cell carcinoma, Ewing sarcoma, myelodysplastic syndrome, myelofibrosis, oral, nasopharyngeal, and peripheral neuroepithelioma.

In certain embodiments the dinucleotide compounds disclosed herein can be given in combination with other anti-cancer agents or cancer therapies. The compounds may be given before, simultaneously, or subsequently with other cancer agents or therapies.

In some embodiments administration of one or more of the disclosed nucleotide compounds are followed by treatment with an immune checkpoint inhibitor. In some embodiments the immune checkpoint inhibitor is a PD-1 inhibitor, PD-L1 inhibitor, and/or a CTLA-4 inhibitor. In other embodiments, the immune checkpoint inhibitor is given in combination with one or more CTLA-4 inhibitors and one or more PD-1 inhibitors or the immune checkpoint inhibitor given in combination with one or more CTLA-4 inhibitors and one or more PD-L1 inhibitors.

In some embodiments the compounds disclosed herein are administered for about 1 to about 5 days per therapeutic cycle.

In yet other embodiments, the checkpoint inhibitor is administered for about 1 to about 3 days per therapeutic cycle.

Also disclosed herein are methods wherein the dinucleotide compounds disclosed herein and the checkpoint inhibitor are administered in combination with a chemotherapeutic agent, a hormonal therapy, a toxin therapy, surgery or combinations thereof.

In some embodiments, the dinucleotides disclosed herein are administered before the checkpoint inhibitor is administered in combination with a chemotherapeutic agent, a hormonal therapy, a toxin therapy, surgery or combinations thereof.

Also disclosed herein are methods of treating a subject who has cancer, comprising administrating to the subject one or more of the dinucleotide compounds disclosed herein preceded by treatment with radiation therapy, wherein the cancer is selected from one or more of breast cancer, prostate cancer, colon cancer, stomach cancer, esophagus, liver, squamous cell cancer, small-cell lung cancer, non-small cell lung cancer, ovarian cancer, cervical cancer, gastrointestinal cancer, pancreatic cancer, glioblastoma, liver cancer, biliary tract, bladder cancer, hepatoma, colorectal cancer, rectal cancer, uterine cancer, cervical cancer, endometrial carcinoma, salivary gland carcinoma, mesothelioma, kidney cancer, vulval cancer who has cancer, pancreatic cancer, thyroid cancer, hepatic carcinoma, testicular cancer, skin cancer, melanoma, brain cancer, neuroblastoma, myeloma, various types of head and neck cancer, acute lymphoblastic leukemia, acute myeloid leukemia, Merkel cell carcinoma, Ewing sarcoma, myelodysplastic syndrome, myelofibrosis, oral, nasopharyngeal, and peripheral neuroepithelioma.

Also disclosed herein are methods of treating a subject who has cancer, comprising administrating to the subject one or more of the dinucleotide compounds disclosed herein followed by treatment with radiation therapy, comprising administrating to the subject a compound of any one of claims 1-96 wherein the cancer is selected from one or more of breast cancer, prostate cancer, colon cancer, stomach cancer, esophagus, liver, squamous cell cancer, small-cell lung cancer, non-small cell lung cancer, ovarian cancer, cervical cancer, gastrointestinal cancer, pancreatic cancer, glioblastoma, liver cancer, biliary tract, bladder cancer, hepatoma, colorectal cancer, rectal cancer, uterine cancer, cervical cancer, endometrial carcinoma, salivary gland carcinoma, mesothelioma, kidney cancer, vulval cancer, pancreatic cancer, thyroid cancer, hepatic carcinoma, testicular cancer, skin cancer, melanoma, brain cancer, neuroblastoma, myeloma, various types of head and neck cancer, acute lymphoblastic leukemia, acute myeloid leukemia, Merkel cell carcinoma, Ewing sarcoma, myelodysplastic syndrome, myelofibrosis, oral, nasopharyngeal, and peripheral neuroepithelioma.

In other embodiments, disclosed herein are methods of treating cancer in a subject comprising administering to said subject one or more of the dinucleotide compounds disclosed herein, followed by treatment with an immune checkpoint inhibitor and radiation therapy, wherein the cancer is selected from one or more of breast cancer, prostate cancer, colon cancer, stomach cancer, esophagus, liver, squamous cell cancer, small-cell lung cancer, non-small cell lung cancer, ovarian cancer, cervical cancer, gastrointestinal cancer, pancreatic cancer, glioblastoma, liver cancer, biliary tract, bladder cancer, hepatoma, colorectal cancer, rectal cancer, uterine cancer, cervical cancer, endometrial carcinoma, salivary gland carcinoma, mesothelioma, kidney cancer, vulval cancer, pancreatic cancer, thyroid cancer, hepatic carcinoma, testicular cancer, skin cancer, melanoma, brain cancer, neuroblastoma, myeloma, various types of head and neck cancer, acute lymphoblastic leukemia, acute myeloid leukemia, Merkel cell carcinoma, Ewing sarcoma, myelodysplastic syndrome, myelofibrosis, oral, nasopharyngeal, and peripheral neuroepithelioma.

In other embodiments, disclosed herein are methods of treating cancer in a subject comprising administering to said subject one or more of the dinucleotide compounds disclosed herein after treatment with radiation, and then followed by treatment with an immune checkpoint inhibitor, wherein the cancer is selected from one or more of breast cancer, prostate cancer, colon cancer, stomach cancer, esophagus, liver, squamous cell cancer, small-cell lung cancer, non-small cell lung cancer, ovarian cancer, cervical cancer, gastrointestinal cancer, pancreatic cancer, glioblastoma, liver cancer, biliary tract, bladder cancer, hepatoma, colorectal cancer, rectal cancer, uterine cancer, cervical cancer, endometrial carcinoma, salivary gland carcinoma, mesothelioma, kidney cancer, vulval cancer, pancreatic cancer, thyroid cancer, hepatic carcinoma, testicular cancer, skin cancer, melanoma, brain cancer, neuroblastoma, myeloma, various types of head and neck cancer, acute lymphoblastic leukemia, acute myeloid leukemia, Merkel cell carcinoma, Ewing sarcoma, myelodysplastic syndrome, myelofibrosis, oral, nasopharyngeal, and peripheral neuroepithelioma.

### BRIEF DESCRIPTION OF THE DRAWINGS

The following drawings form part of the present specification and are included to further demonstrate certain aspects or embodiments of the present disclosure. The disclosure may be better understood by reference to one or more of these drawings in combination with the detailed description of specific embodiments presented herein.

FIG. 1 shows the general structure of the claimed compounds with free 5′-hydroxyl group and X is O or S.

FIG. 2 shows the general structure of the claimed compounds with a derivatized 5′-hydroxyl group.

FIG. 3 shows the general structure of the claimed compounds with a free 3′-hydroxyl group. All molecules are linear, non-cyclic, dinucleotides.

FIG. 4 shows the general structure of the claimed compounds with a free 5′-hydroxyl group.

FIG. 5A-5B—5A shows compound 11 and its two enantiomers—compound 11-Rp and compound 11-Sp; 5B shows compound 12 and its two enantiomers compound 12-Rp and compound 12-Sp.

FIG. 6A-6E shows the effect of compounds 1-17 on cell viability of LLC and MC38 cells. 6A shows the effect of compounds 1-4 on cell viability of LLC and MC38 cells. 6B shows the effect of compounds 5-8 on cell viability of LLC and MC38 cells. 6C shows the effect of compounds 9-12 on cell viability of LLC and MC38 cells. 6D shows the effect of compounds 13-16 on cell viability of LLC and MC38 cells. 6E show the effect of compound 17 on cell viability of LLC and MC38 cells.

FIG. 7A-7B—shows the specificity of the tested dinucleotides toward telomerase-negative normal non-cancerous human BJ fibroblasts. 7A shows the effect of compounds 1-6 and 11 and 12 on cell viability on non-cancerous/telomerase negative BJ cells. 7B shows the effect of compound 13 on cell viability on non-cancerous/telomerase negative BJ cells.

FIG. 8A-8C—H22 Murine Model for Hepatocellular Carcinoma (Syngeneic Mouse Model); FIG. 8A shows Tumor volume (mm³) for BALB/c mice inoculated with H22 tumor cells and treated with vehicle; MAIA-2022-12; MAIA-2021-20; Anti-PD1; MAIA-2022-12+Anti-PD-1 or MAIA-2021-20+Anti-PD-1. FIG. 8B shows Body weight change in the treated mice. FIG. 8C shows individual data from the H22 experiment.

FIG. 9A-9C—M38 Murine Model for Colorectal Carcinoma (Syngeneic Mouse Model); FIG. 9A shows Tumor volume (mm³) for C57Bl/6N mice inoculated with MC38 tumor cells and treated with vehicle; MAIA-2022-12; MAIA-2021-20; Anti-PD1; MAIA-2022-12+Anti-PD-1 or MAIA-2021-20+Anti-PD-1. FIG. 9B shows Body weight change in the treated mice. FIG. 9C shows individual data from the M38 experiment.

FIG. 10A-10C—YUMM1.7 Murine Model for Melanoma (Syngeneic Mouse Model). FIG. 10A shows Tumor volume for c57BL/6N mice inoculated with YUMM1.7 tumor cells and treated with vehicle, MAIA-2022-12 or MAIA-2021-20; FIG. 10B shows body weight change for mice treated with vehicle, MAIA-2022-12 or MAIA-2021-20; FIG. 10(c) shows individual data from the YUMM1.7 experiment.

FIG. 11A-11E—YUMM1.7 Rechallenge Studies. FIG. 11A shows tumor volume in mice after rechallenge with control, MAIA-2022-12, or MAIA-2021-020. FIG. 11B shows Body weight after rechallenge with control, MAIA-2022-12, or MAIA-2021-020; Fig. C-E shows individual data from the control, MAIA-2022-12 and MAIA-2021-20 tumor volume experiments.

### DETAILED DESCRIPTION OF THE EMBODIMENTS

Disclosed herein are dinucleotide compounds that target and modify telomeric structures of cancer cells. The compounds are all linear, non-cyclic, dinucleotides.

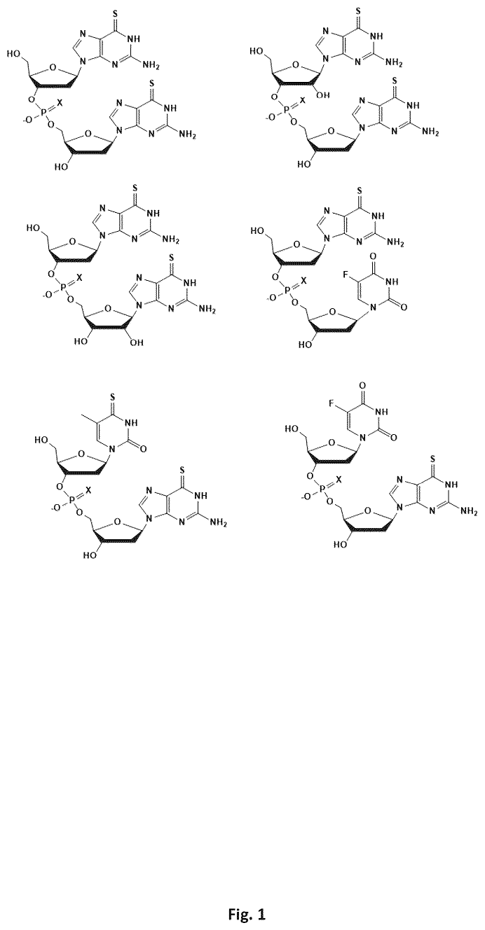

In aspects of the disclosure, the compounds disclosed herein are dinucleotide molecules that contain modified nucleosides which are converted in cells into human telomerase substrates, for example, nucleoside-5′-triphosphates. The nucleotides may be connected via phosphodiester group, (relatively fast cleavable linkage, Rp), or via phosphorothioate group (relatively slow cleavable linkage, Sp). In certain embodiments of this aspect, the general structure of the disclosed compounds with free 5′-hydroxyl group molecules are linear, non-cyclic, dinucleotides. The nucleoside-connecting 5′-3′ internucleoside linkage is either phosphodiester, (where X═O), or phosphorothioate, (where X═S). The dinucleotide-composing nucleosides consist of 6-thio-2′-deoxyguanosine, 6-thioguanosine, 4-thio-thimidine, 5-fluorouridine When X═S, either a mixture of two Rp- and Sp-isomers, or individual single Rp- or Sp-isomer (FIG. 1).

In some embodiments of the above aspect, the dinucleotide compounds comprise the structure of Formula I or a pharmaceutically acceptable salt thereof.

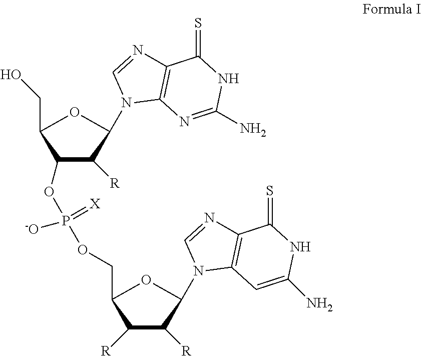

wherein R is independently H or OH and X is O or S. In some embodiments X is O. In other embodiments X is S.

When X is O the dinucleotide compounds of Formula I comprise the following structures or pharmaceutically acceptable salts thereof:

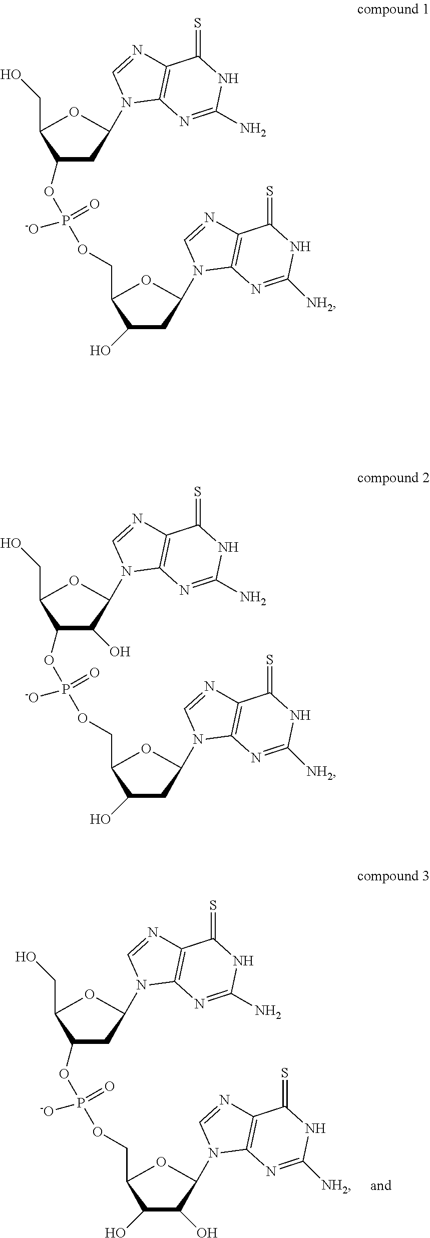

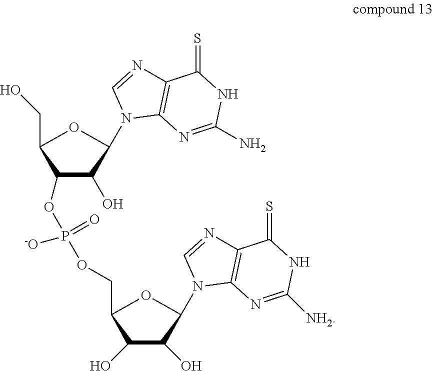

When X is S the dinucleotide compounds of Formula I comprise the following structures or pharmaceutically acceptable salts thereof:

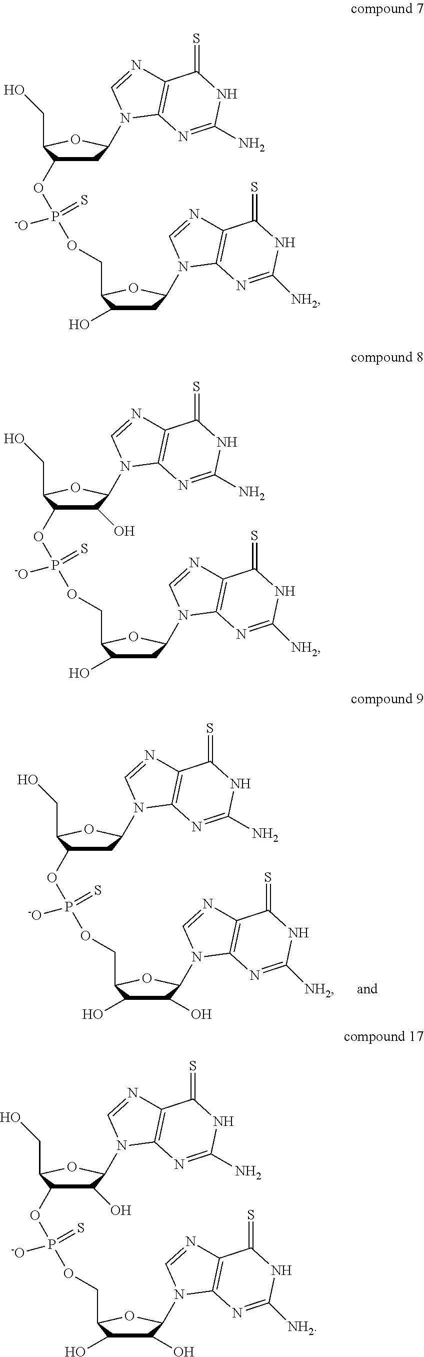

In yet other embodiments the dinucleotide compounds comprise the following structures or pharmaceutically acceptable salts thereof

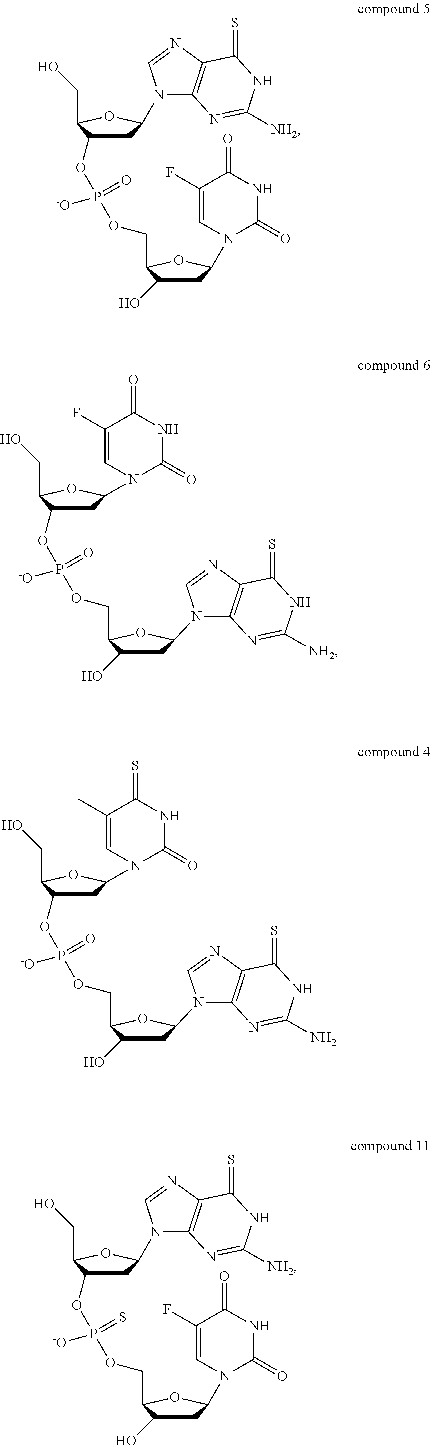

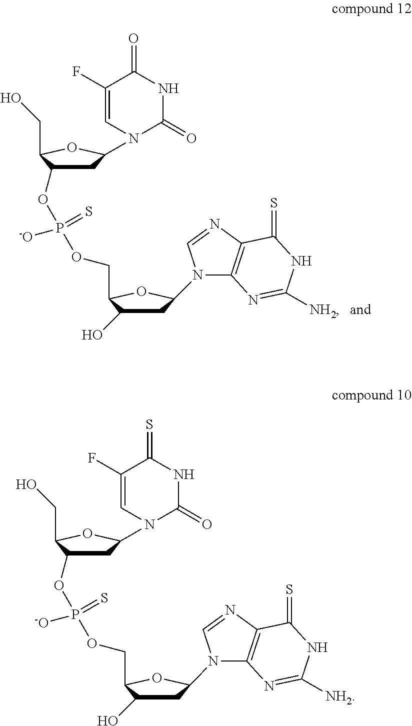

Also disclosed herein are enantiomers of compound 11 that have the following stereochemistry:

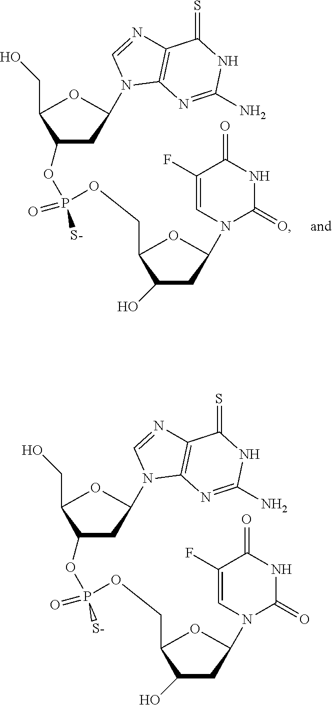 [SMILES: Nc1nc2c(ncn2[C@H]2C[C@@H](O[P@](=O)([S-])OC[C@H]3O[C@@H](n4cc(F)c(=O)[nH]c4=O)C[C@H]3O)[C@@H](CO)O2)c(=S)[nH]1 ; Nc1nc2c(ncn2[C@H]2C[C@@H](O[P@](=O)([S-])OC[C@H]3O[C@@H](n4cc(F)c(=O)[nH]c4=O)C[C@H]3O)[C@@H](CO)O2)c(=S)[nH]1]

In another embodiment are enantiomers of compound 12 that have the following stereochemistry:

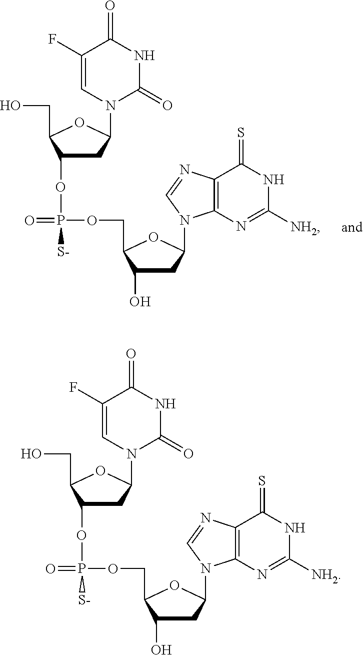

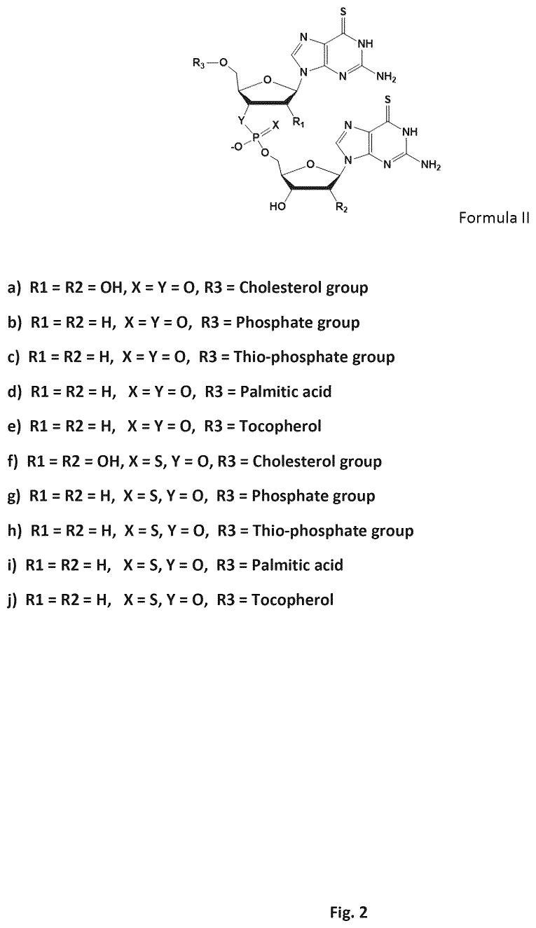

In another aspect, disclosed herein are dinucleotide compounds of Formula II or pharmaceutically acceptable salts thereof with a derivatized 5′-hydroxyl group, such as without limitation 5′-conjugated lipid, 5′-phosphate, or 5′-thio-phosphate groups (FIG. 2).

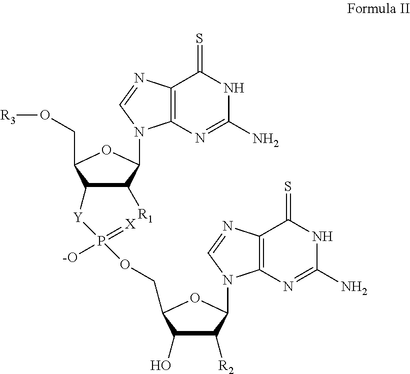

wherein R₁ and R₂ are independently H or OH;
X is O or S;
Y is O; and
R₃ is selected from the group consisting of a phosphate group, thio-phosphate group, palmitic acid, tocopherol, and cholesterol group.

In some embodiments of Formula II, R₁ and R₂ are independently H or OH, X is O, Y is O, and R₃ is a cholesterol group. In some embodiments, the dinucleotide compounds of Formula II have the following structures,

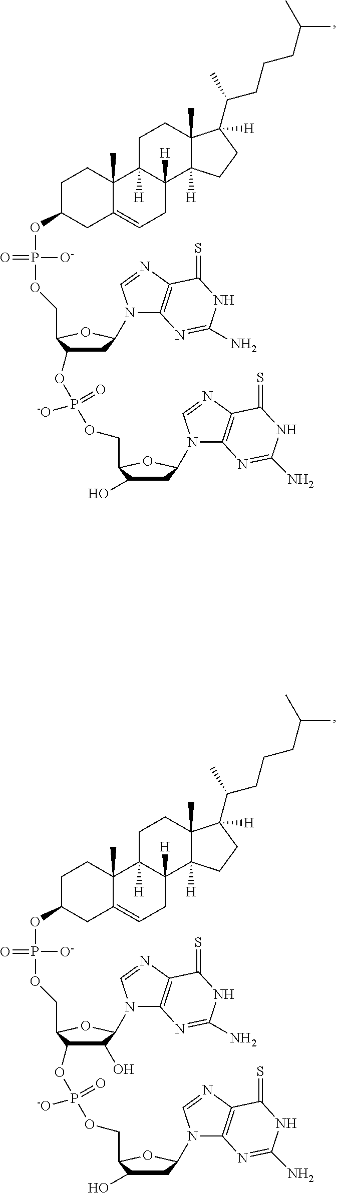

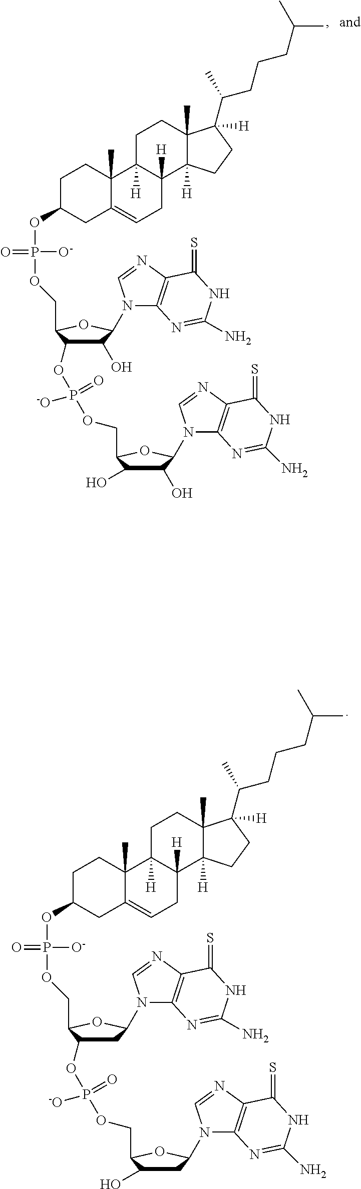

In some embodiments of Formula II, R₁ and R₂ are independently H or OH, X and Y are O, and R₃ is a phosphate group. In some embodiments, the dinucleotide compounds of Formula II have the following structures:

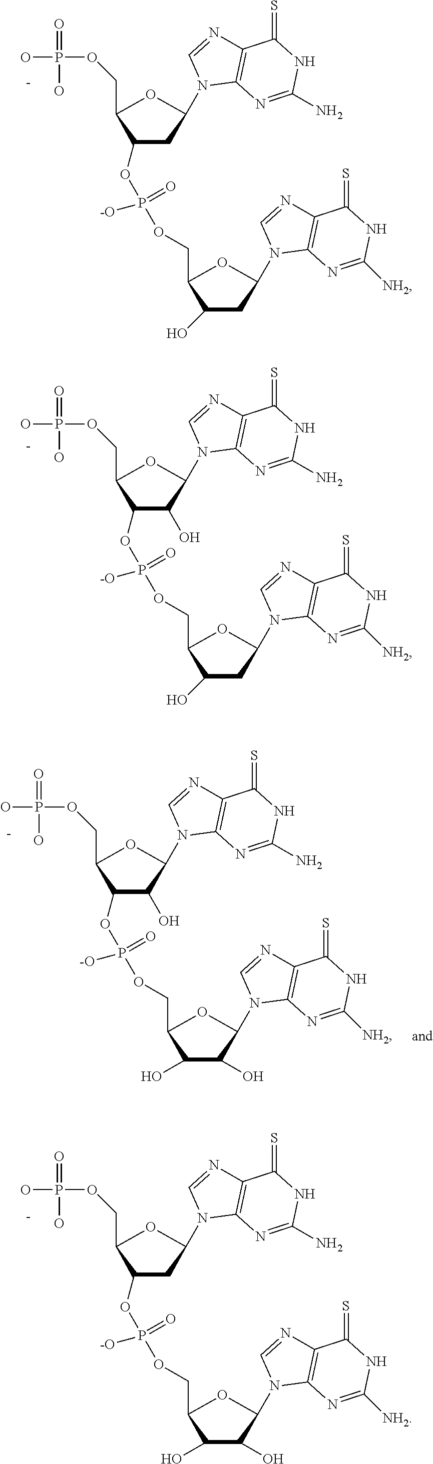

In other embodiments of Formula II, R₁ and R₂ are independently H or OH, X and Y are independently O, and R₃ is a thio-phosphate group. In some embodiments, the dinucleotide compounds of Formula II have the following structures:

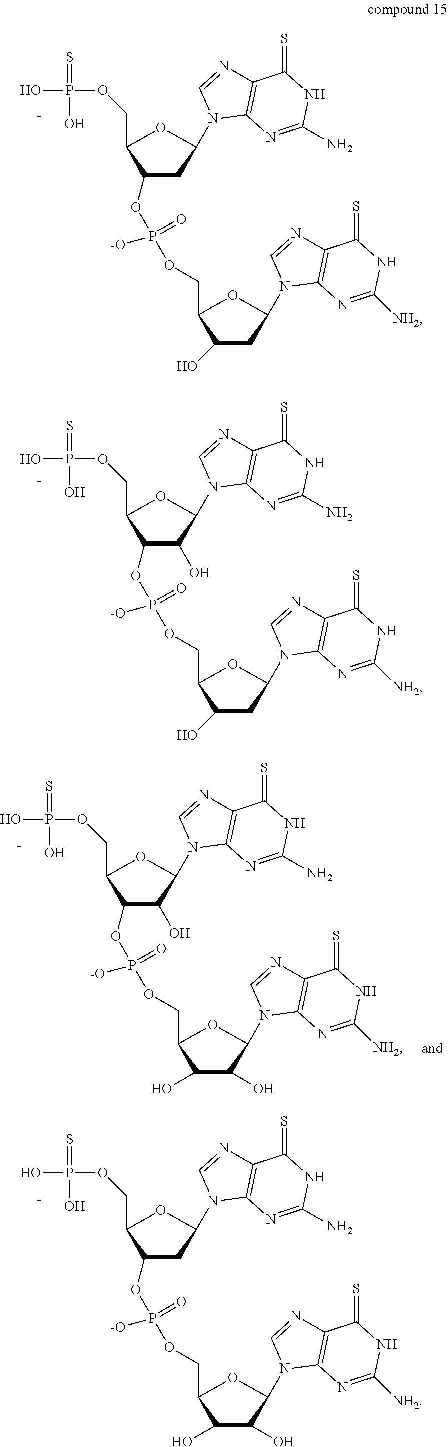

In yet other embodiments of Formula II, R₁ and R₂ are independently H or OH; X and Y are independently O, and R₃ is palmitic acid. In some embodiments, the dinucleotide compounds of Formula II have the following structures:

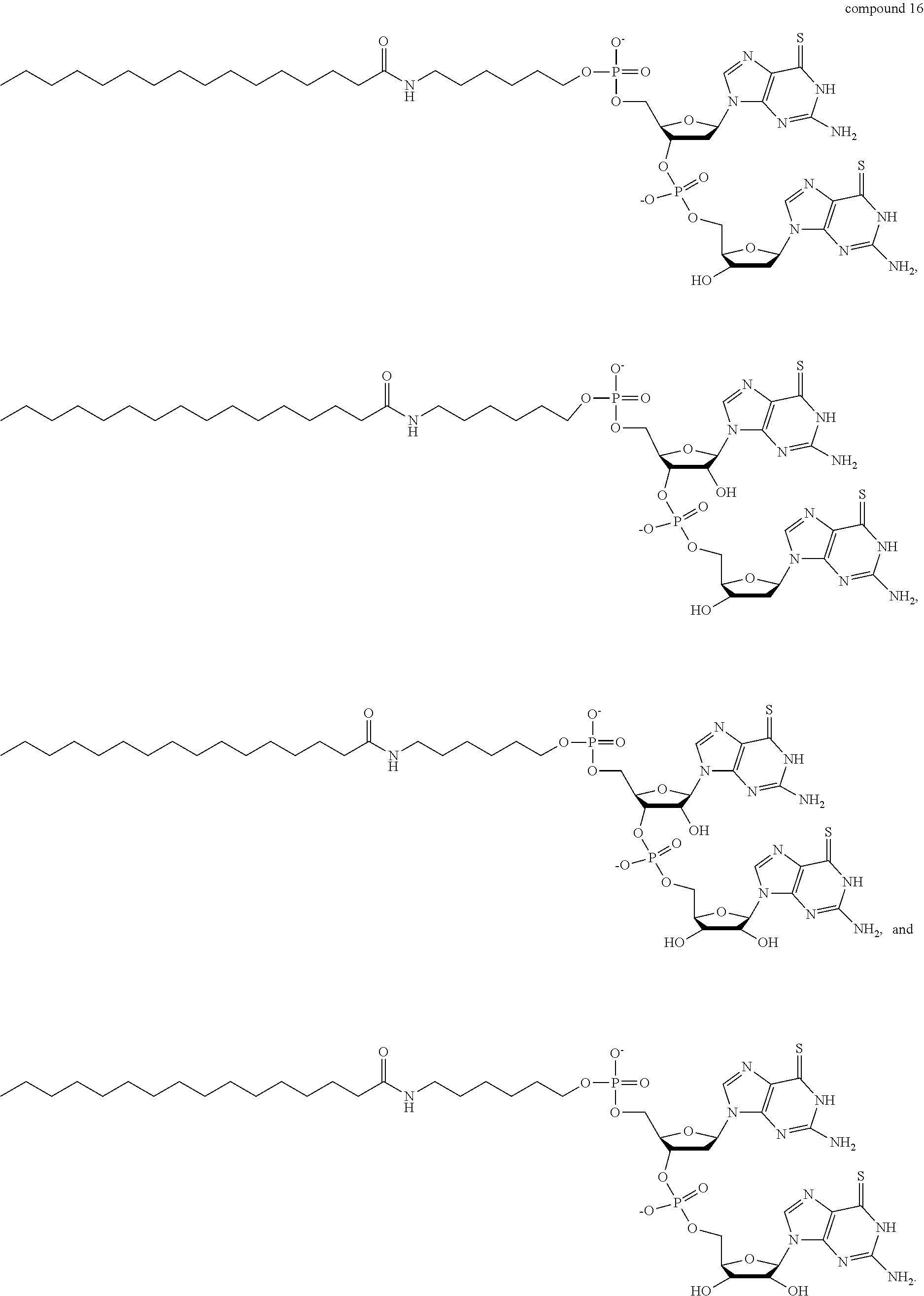

In yet other embodiments of Formula II, R₁ and R₂ are independently H or OH, X and Y are independently O, and R₃ is tocopherol. In some embodiments, the dinucleotide compounds of Formula II have the following structures:

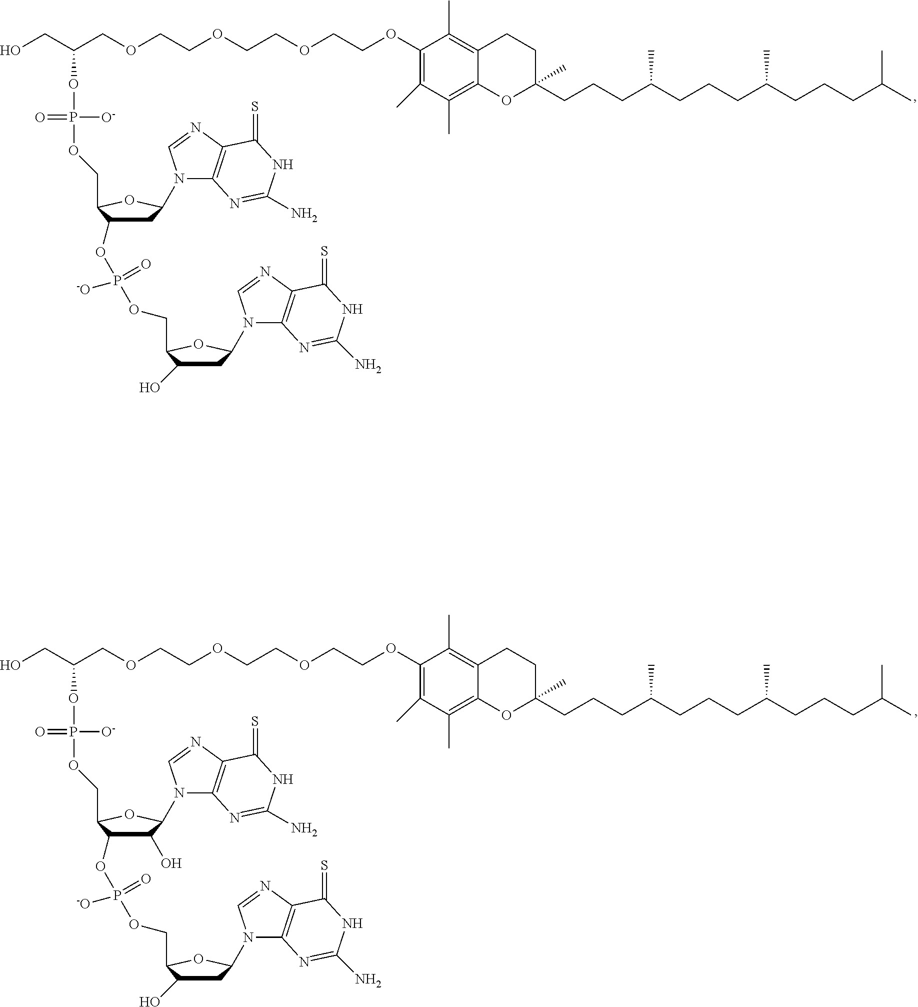

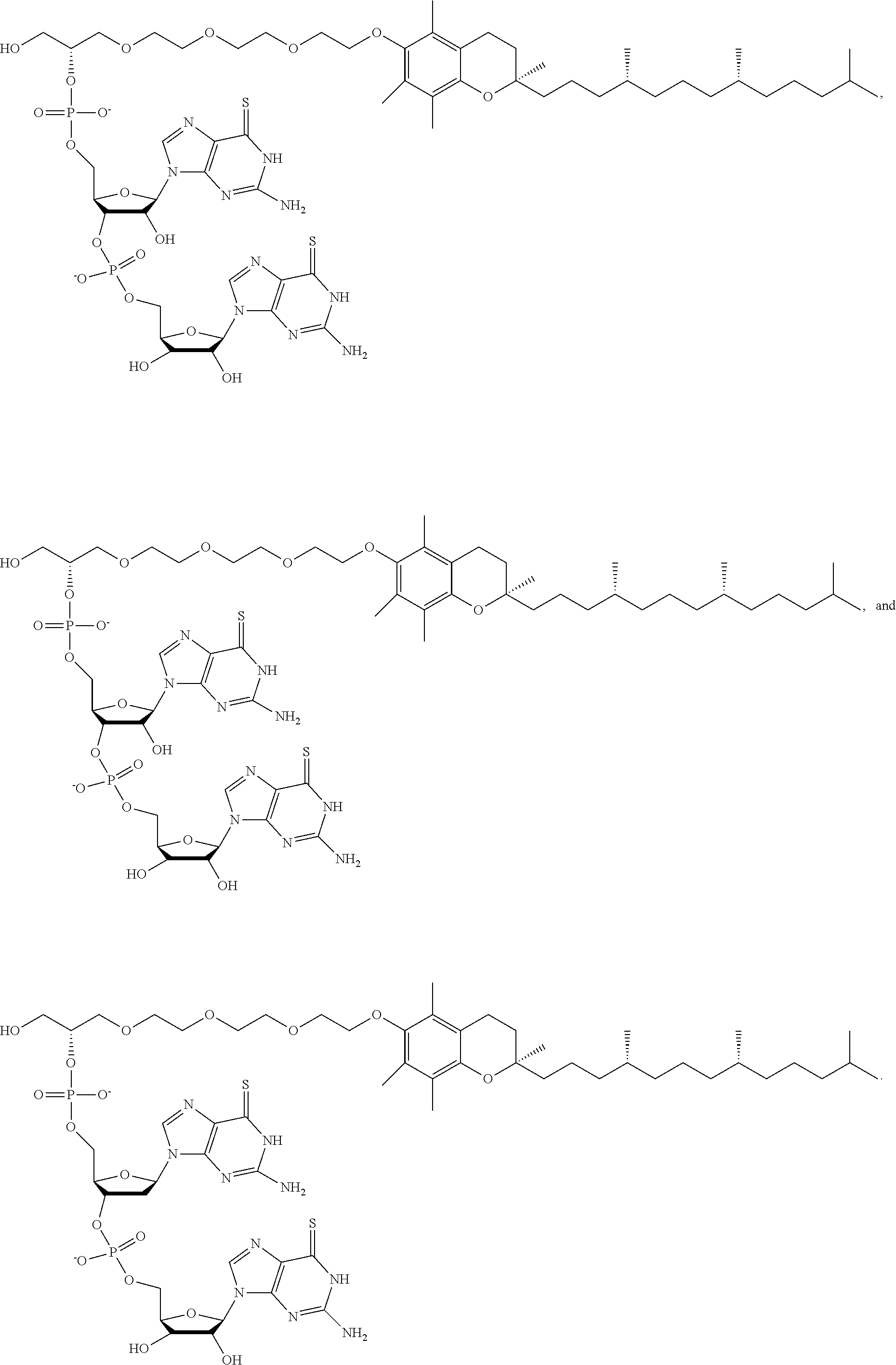

In other embodiments of Formula II, R₁ and R₂ are independently H or OH, X is S, Y is O, and R₃ is a cholesterol group. In some embodiments, the dinucleotide compounds of Formula II have the following structures:

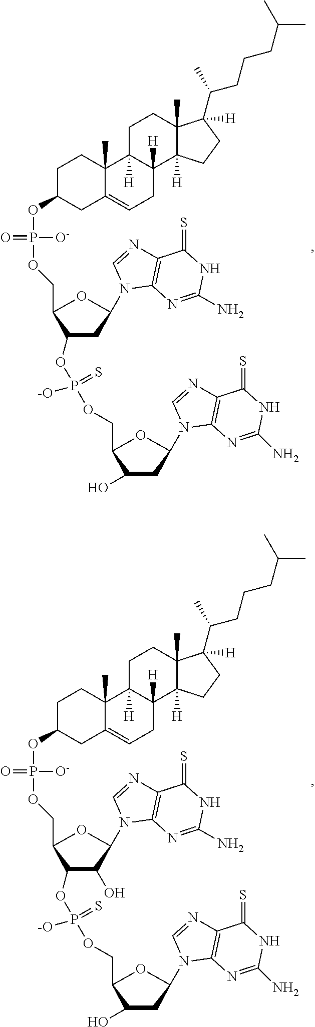

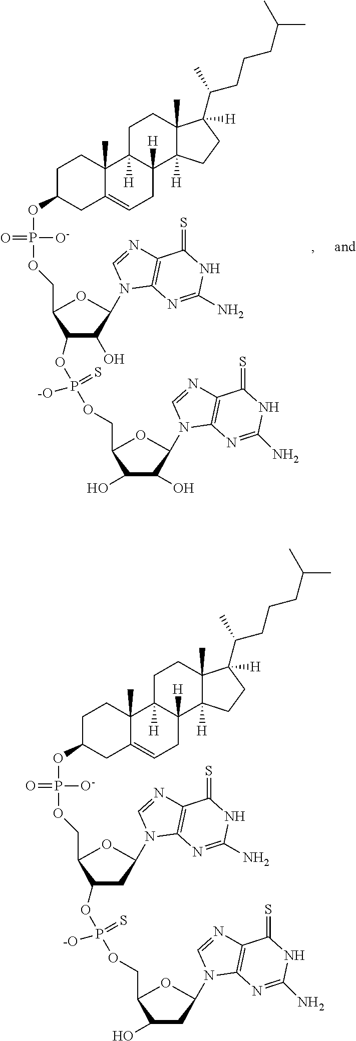

In other embodiments of Formula II, R₁ and R₂ are independently H or OH, X is S, Y is O, and R₃ is a phosphate group. In some embodiments, the dinucleotide compounds of Formula II have the following structures:

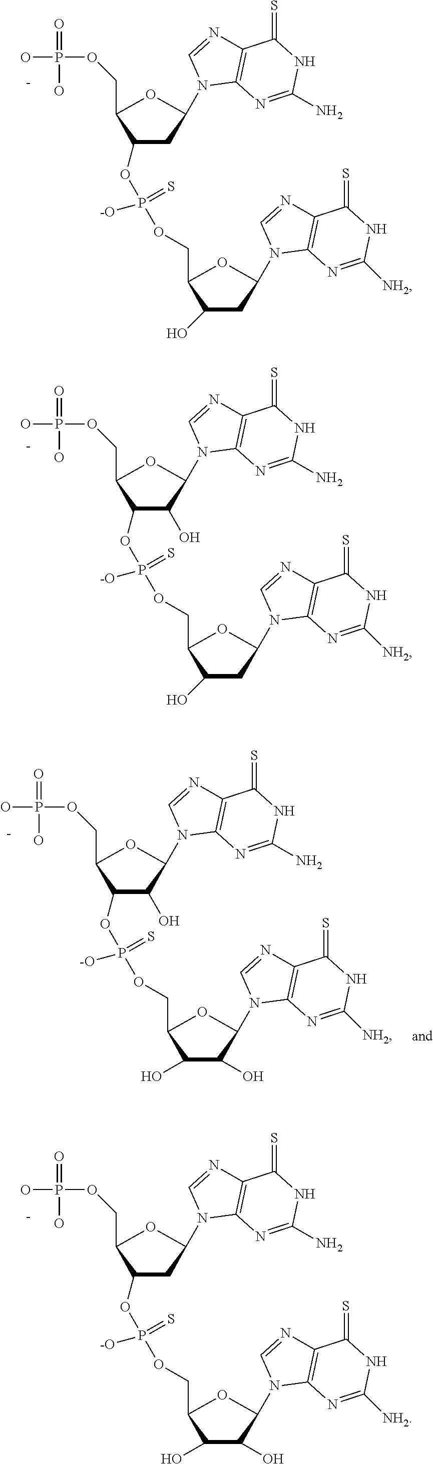

In some embodiments of Formula II, R₁ and R₂ are independently H or OH, X is S, Y is O, and R₃ is a thio-phosphate group. In some embodiments, the dinucleotide compounds of Formula II have the following structures:

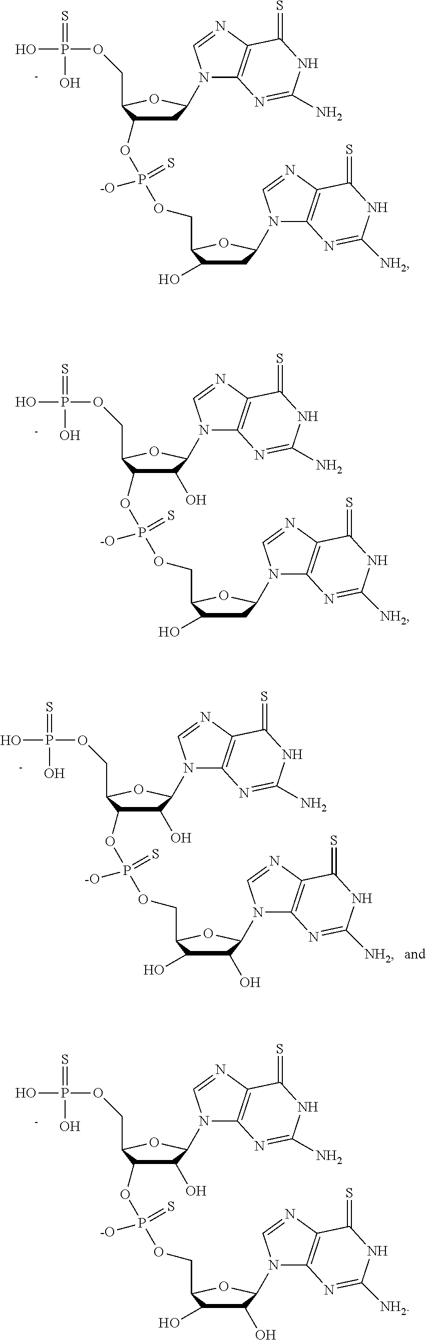

In some embodiments of Formula II, R₁ and R₂ are independently H or OH, X is S, Y is O, and R₃ is palmitic acid. In some embodiments, the dinucleotide compounds of Formula II have the following structures:

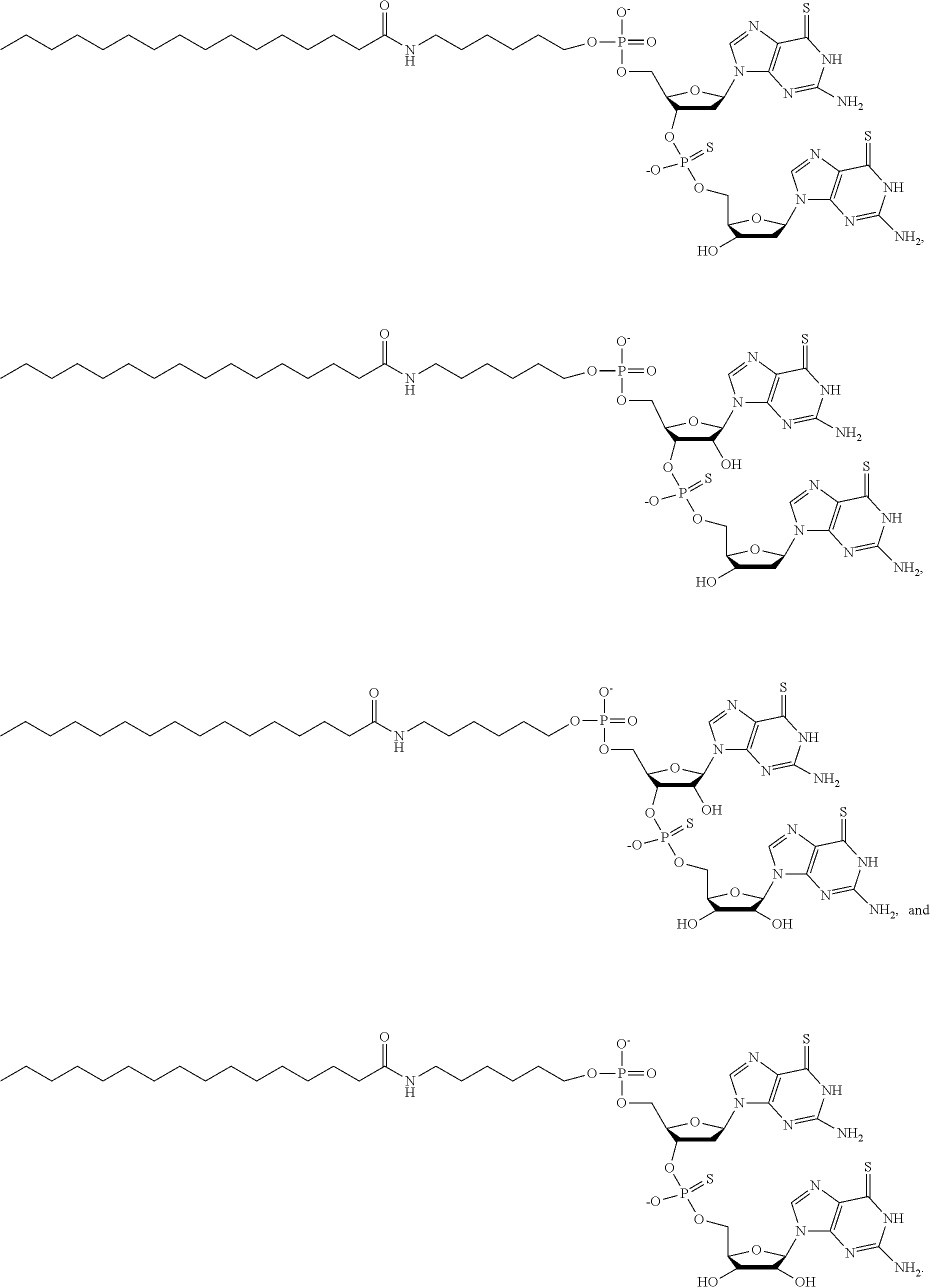

In other embodiments of Formula II, R₁ and R₂ are independently H or OH, X is S, Y is O, and R₃ is tocopherol. In some embodiments, the compounds of Formula II have the following structures:

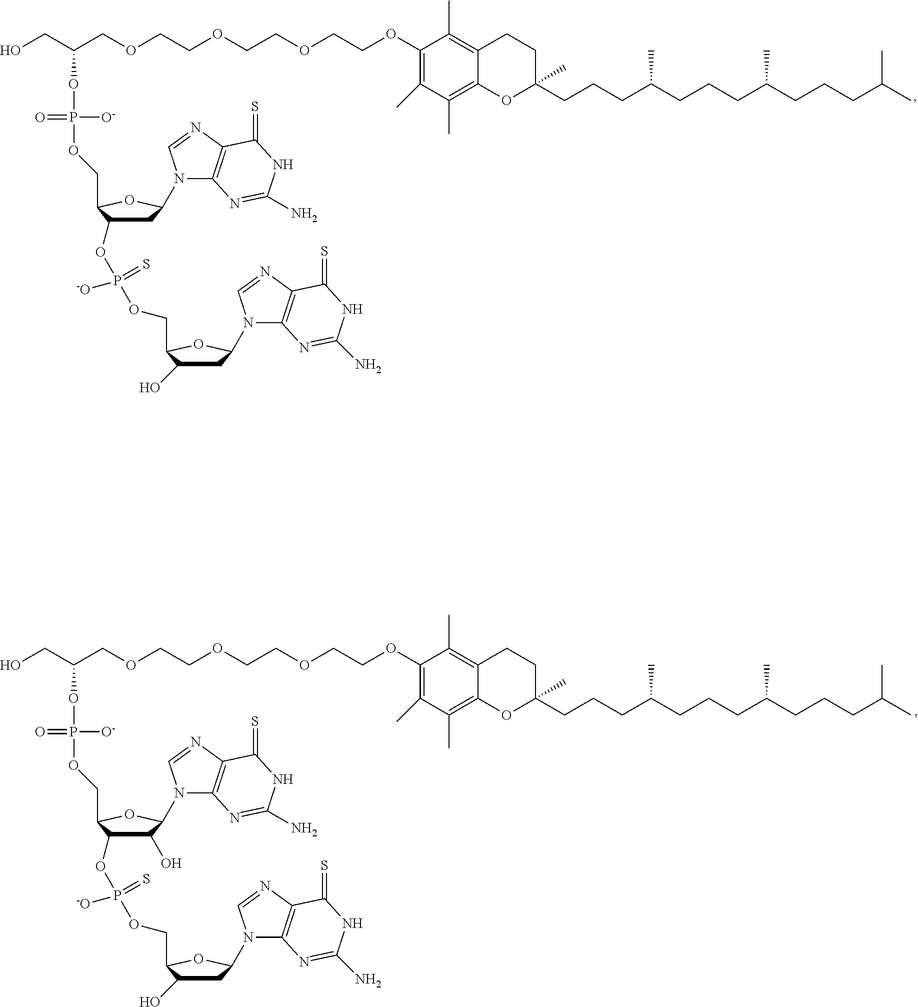

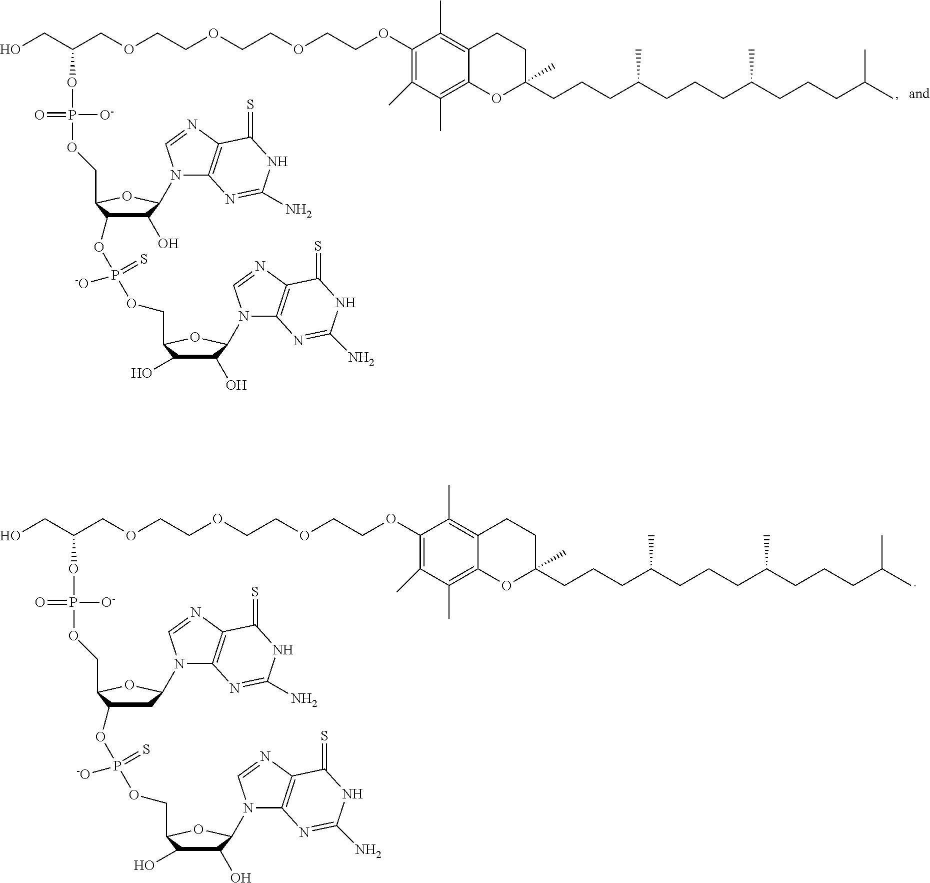

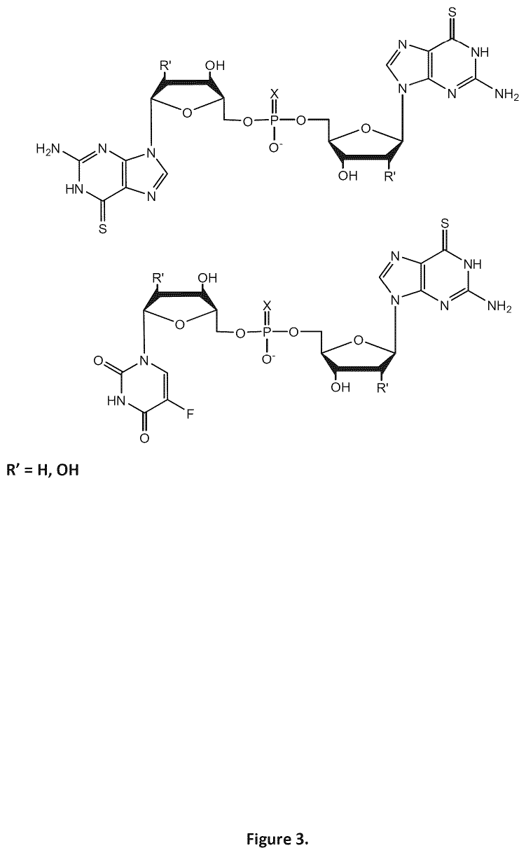

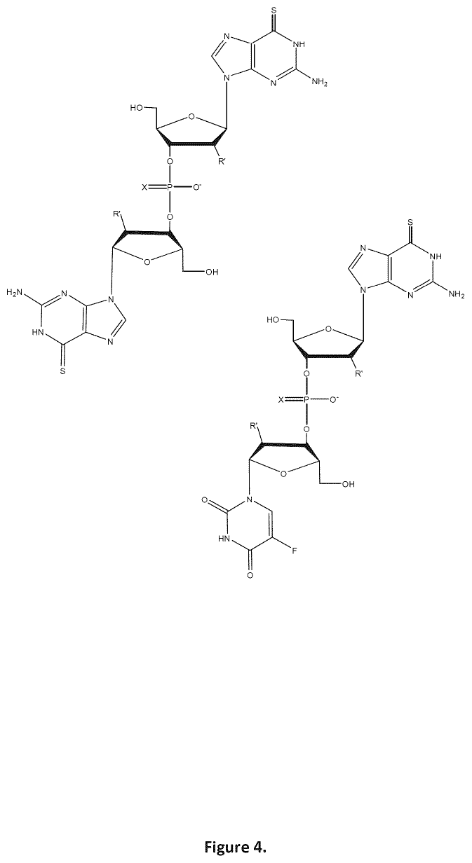

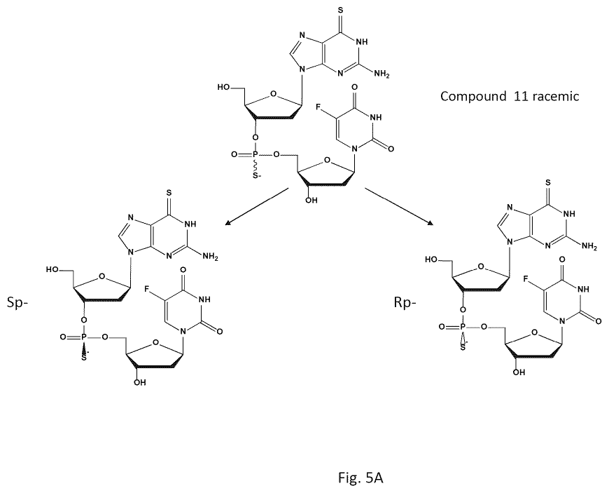

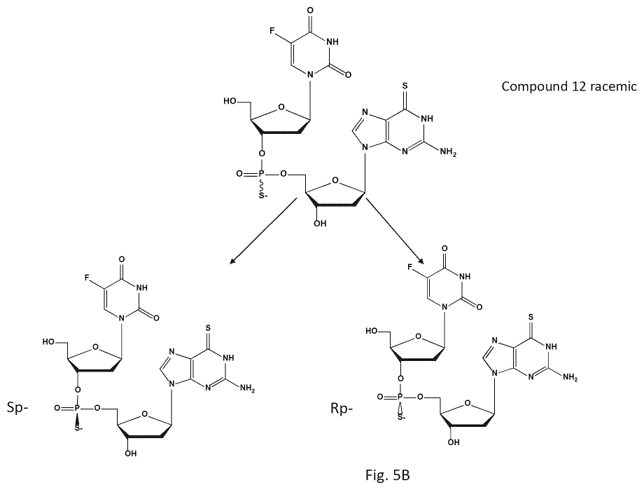

In certain aspects, the structure of the disclosed dinucleotide compounds have a free 3′-hydroxyl group. All molecules are linear, non-cyclic, dinucleotides. The nucleoside-connecting 5′-5′ internucleoside linkage is either phosphodiester, (where X═O), or phosphorothioate, (where X═S). The dinucleotide-composing nucleosides consist of 6-thio-2′-deoxyguanosine, 6-thioguanosine, and 5-fluorouridine. When X═S, either a mixture of two Rp-and Sp-isomers, or individual single Rp- or Sp-isomer (FIG. 3).

In embodiments of this aspect are dinucleotide compounds comprising the structure of Formula III or pharmaceutically acceptable salts thereof:

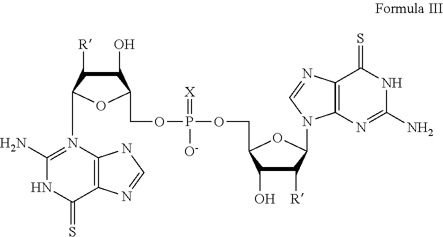

wherein X is O or S and R′ is independently H or OH. In some embodiments, the dinucleotide compounds of Formula III have the following structures:

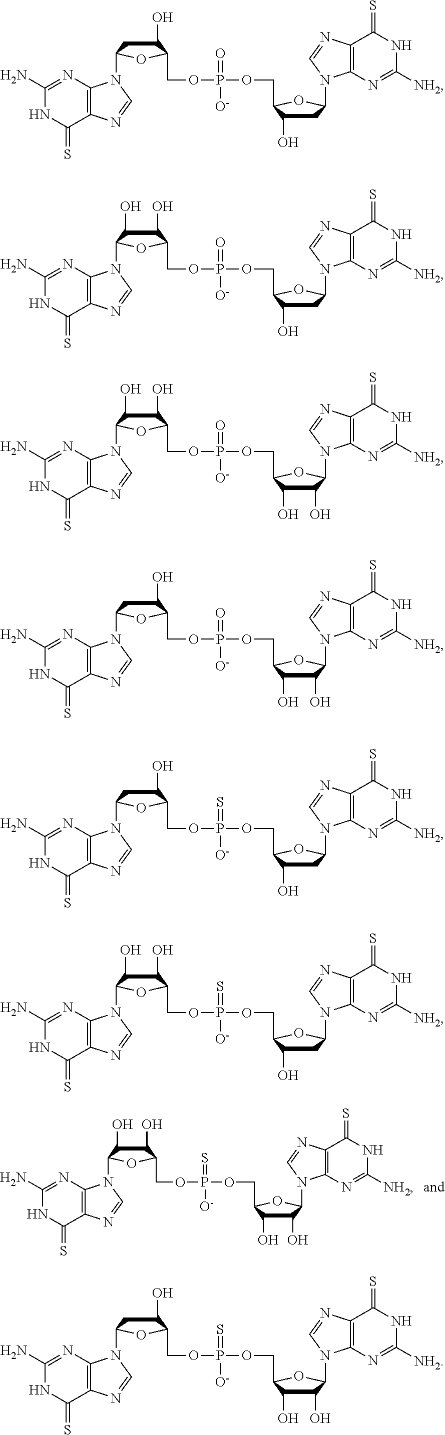

In other embodiments are dinucleotide compounds comprising the structure of Formula IV.

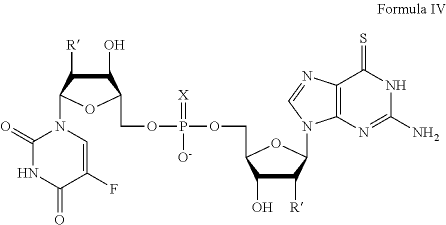

wherein X is O or S and R′ is independently H or OH.

In some embodiments, the dinucleotide compounds of Formula IV have the following structures:

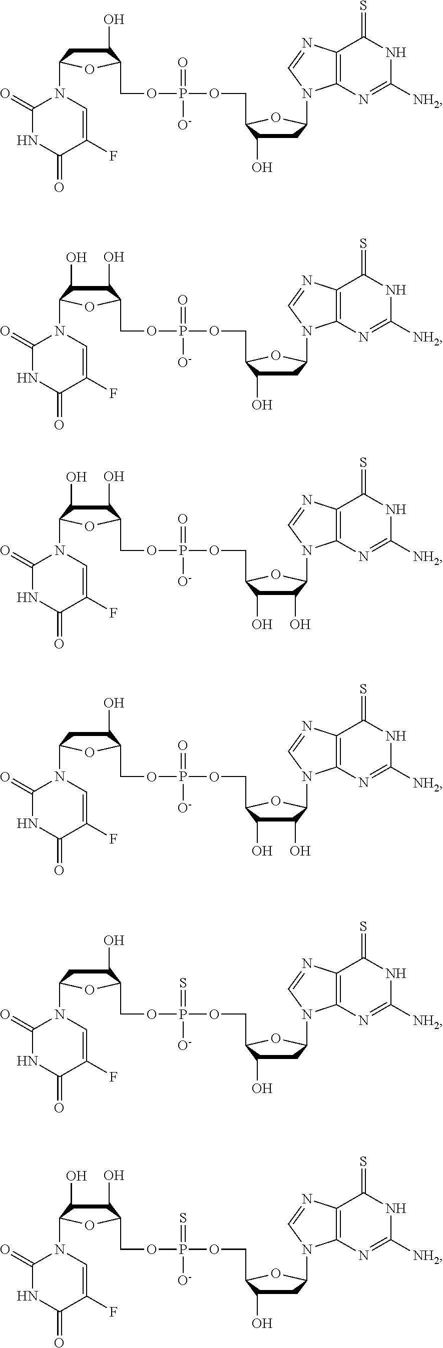

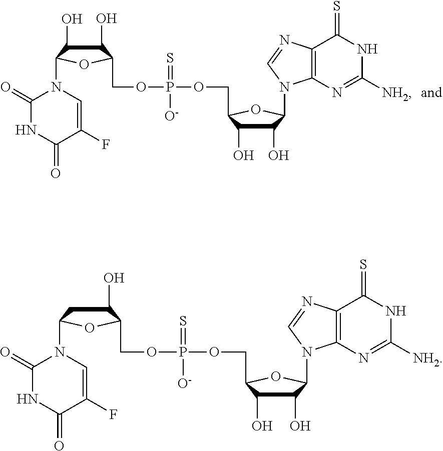

In other aspects, the general structure of the disclosed dinucleotide compounds have free 5′-hydroxyl group. All molecules are linear, non-cyclic, dinucleotides. The nucleoside-connecting 3′-3′ internucleoside linkage is either phosphodiester, (where X═O), or phosphorothioate, (where X═S). The dinucleotide-composing nucleosides consist of 6-thio-2′-deoxyguanosine, 6-thioguanosine, and 5-fluorouridine. When X═S, either a mixture of two Rp-and Sp-isomers, or individual single Rp- or Sp-isomer (FIG. 4).

Compounds representative of FIG. 4 are dinucleotide compounds comprising the structure of Formula V or pharmaceutically acceptable salts thereof, wherein R′ is independently H or OH:

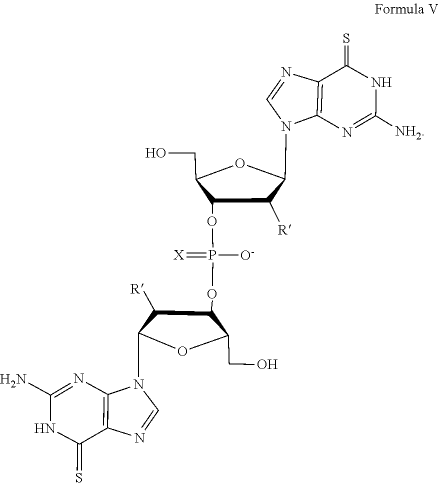

In yet other embodiments representative of FIG. 4 are dinucleotide compounds comprising the structure of Formula VI, or pharmaceutically acceptable salts thereof wherein R′ is independently H or OH:

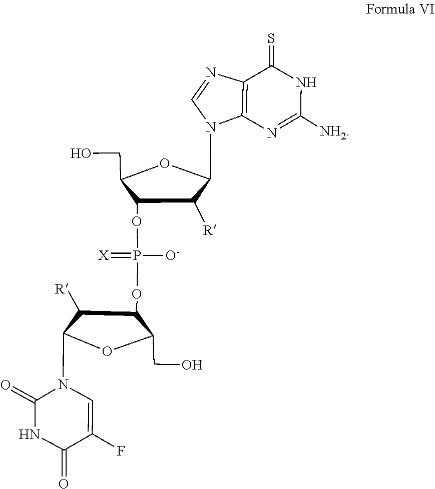

In other embodiments are dinucleotide compounds comprising the structure of Formula VII wherein R₁ or R₂ are independently H or OH and X is S or O.

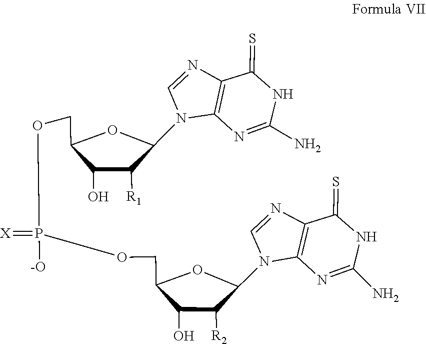

In some embodiments, the dinucleotide compounds of Formula VII have the following structures wherein X is O and R₁ and R₂ are H or OH:

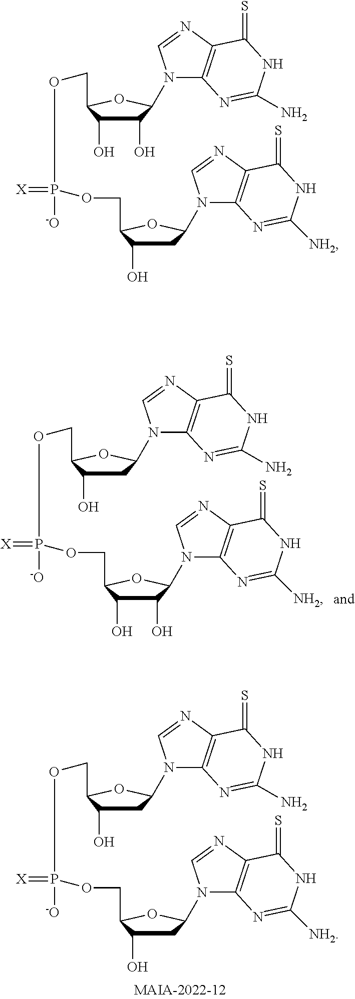

In some embodiments, the dinucleotide compounds of Formula VII have the following structures wherein X is S and R₁ and R₂ are H or OH:

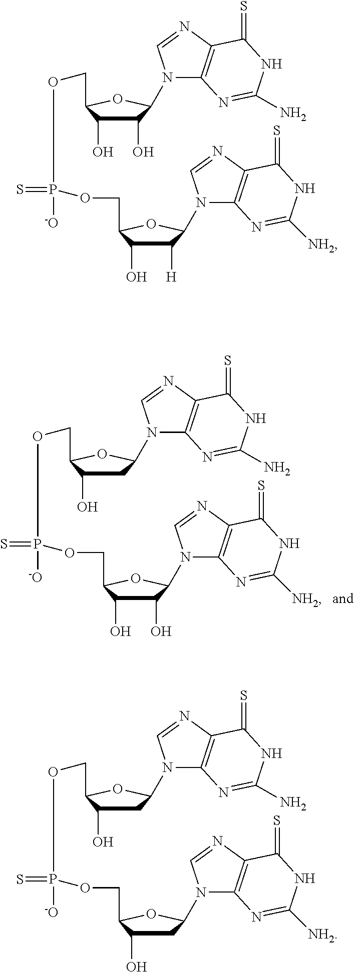

One aspect of the disclosure comprises a method for treating a cancer including a resistant, refractory and/or metastatic cancer, the method comprising administering to the subject a first amount or dose of one or more dinucleotide compounds disclosed herein in a therapeutically effective amount that is effective to shorten telomere length; reduce size of a tumor; reduce growth rate of a tumor; reduce incidence of metastasis; eliminating metastasis, promote an immune response; reduce progression of the cancer; increase lifespan of the subject; or a combination thereof.

In embodiments of this aspect, the dinucleotide compounds disclosed herein are given to a subject in need thereof in combination with one or more other cancer therapies wherein the combination is effective to shorten telomere length; reduce size of a tumor; reduce growth rate of a tumor; reduce incidence of metastasis; promote an immune response; reduce progression of the cancer; increase lifespan of the subject; or a combination thereof.

The term “subject” as used herein refers to either a human or non-human, such as primates, mammals, and vertebrates. According to some embodiments, the subject is a human.

The term “subject in need of such treatment or in need thereof” as used herein refers to (i) a patient who suffers from a cancer; and (ii) a subject who will be administered a dinucleotide compound or dinucleotide compounds as disclosed herein.

The term “therapeutically effective dose” as used herein refers to a dose (i.e., dose and frequency of administration) that eliminates, reduces, or prevents the progression of a particular disease manifestation in a percentage of a population. An example of a commonly used therapeutic component is the ED₅₀, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect also may include, directly or indirectly, the arrest reduction or elimination of the progression of a disease manifestation.

The terms “therapeutically effective amount”, an “amount effective”, or “pharmaceutically effective amount” of an active agent are used interchangeably to refer to an amount that is sufficient to provide the intended benefit of treatment. An effective amount of an active agent that can be employed according to the described invention generally ranges from generally about 0.01 mg/kg body weight to about 100 g/kg body weight. However, dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods.

The term “treat” or “treating” includes abrogating, substantially inhibiting, slowing or reversing the progression of a disease, condition or disorder, substantially ameliorating clinical symptoms of a condition, substantially preventing the appearance of clinical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

According to some embodiments, the cancer treated using the dinucleotide compounds disclosed herein is, without limitation, a carcinoma, a sarcoma, a leukemia, a lymphoma/myeloma or a brain/spinal cord cancer. According to some embodiments, the cancer comprises a solid tumor comprising tumor cells, a metastatic cancer comprising metastatic tumor cells, or a combination thereof.

In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, prostate, skin, stomach, testis, tongue, or uterus. A tumor can comprise a malignant or benign growth.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odonto sarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Anticancer activity of the dinucleotides, (formulated and administered in PBS solutions), was evaluated in vitro in various cancer cell lines, according to a commonly used procedures for assessment of compounds cytotoxicity, as out lined in the following publications: Mender, I., Gryaznov, S., Dikmen, Z. G., Wright, W. E., & Shay, J. W. (2015). Induction of telomere dysfunction mediated by the telomerase substrate precursor 6-thio-2′-deoxyguanosine. Cancer Discovery, 5, 82-95; Mender et al., (2020). Telomere Stress Potentiates STING-Dependent Anti-tumor Immunity. Cancer Cell, 38, 400-411.

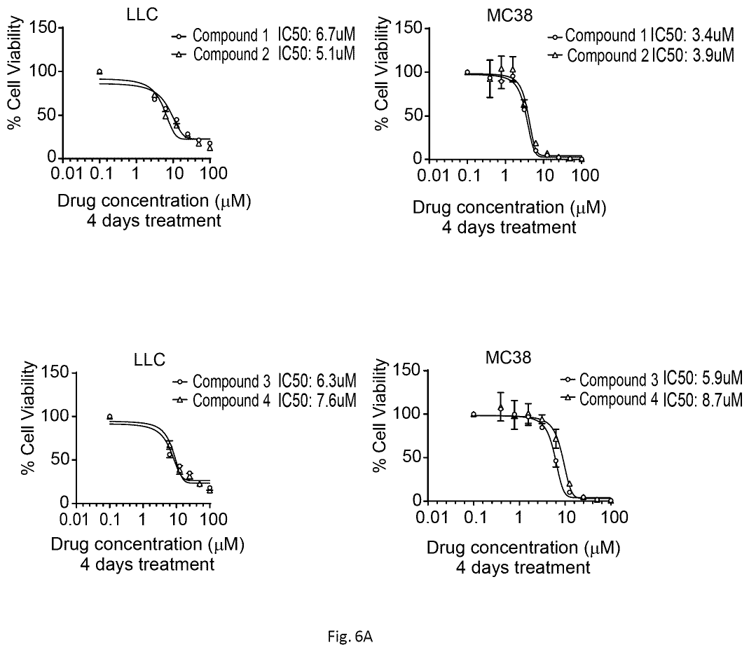

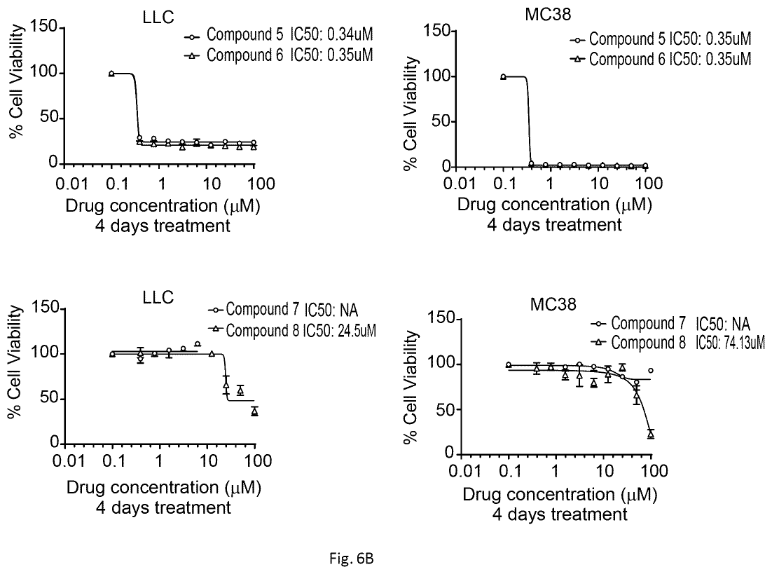

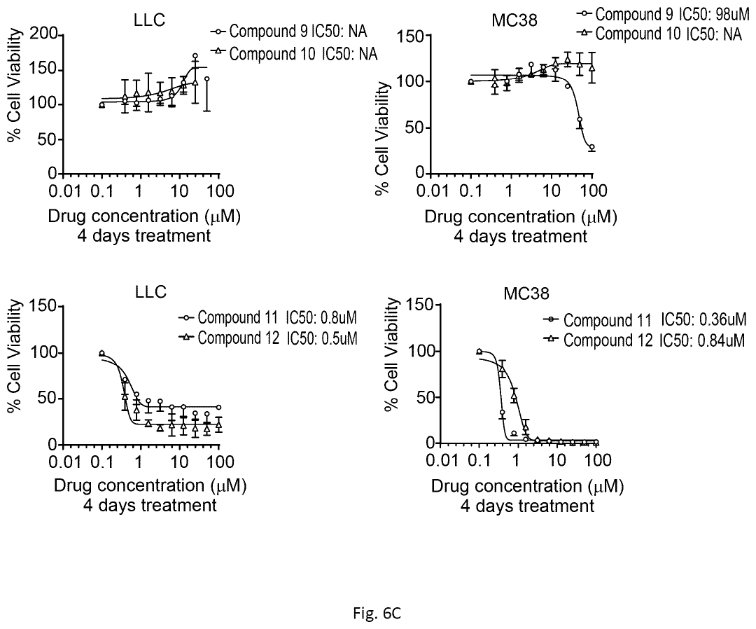

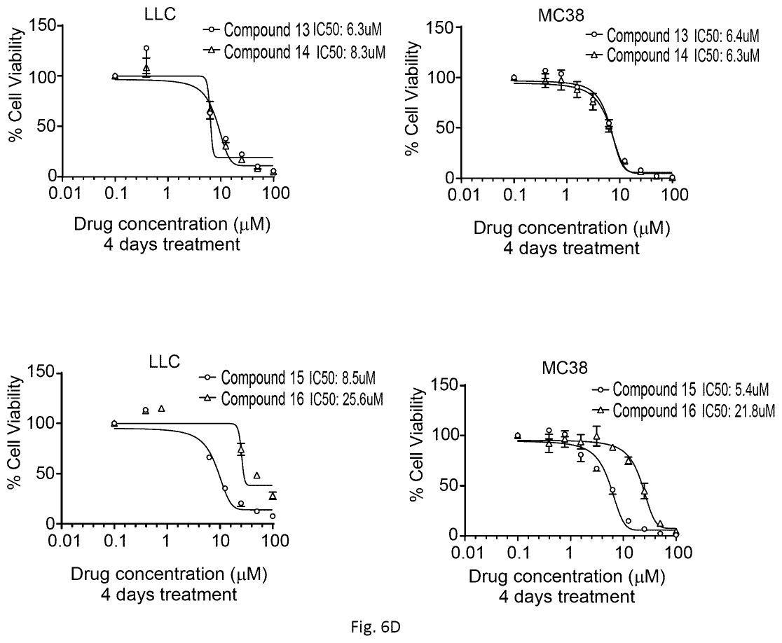

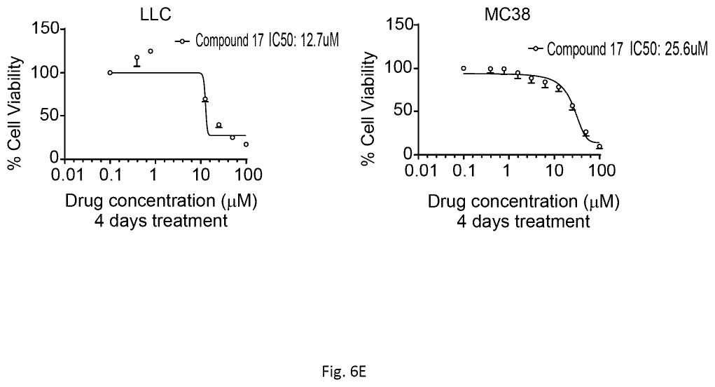

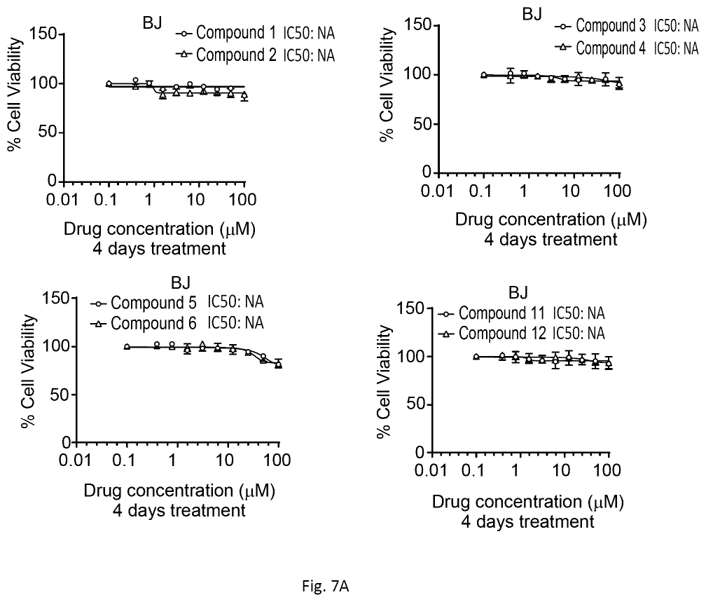

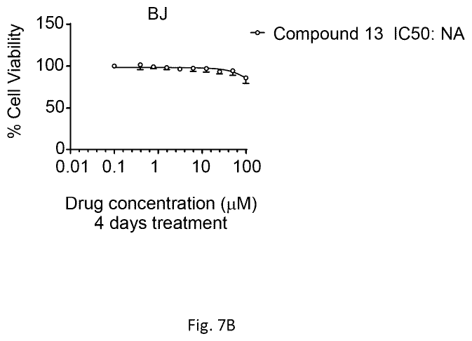

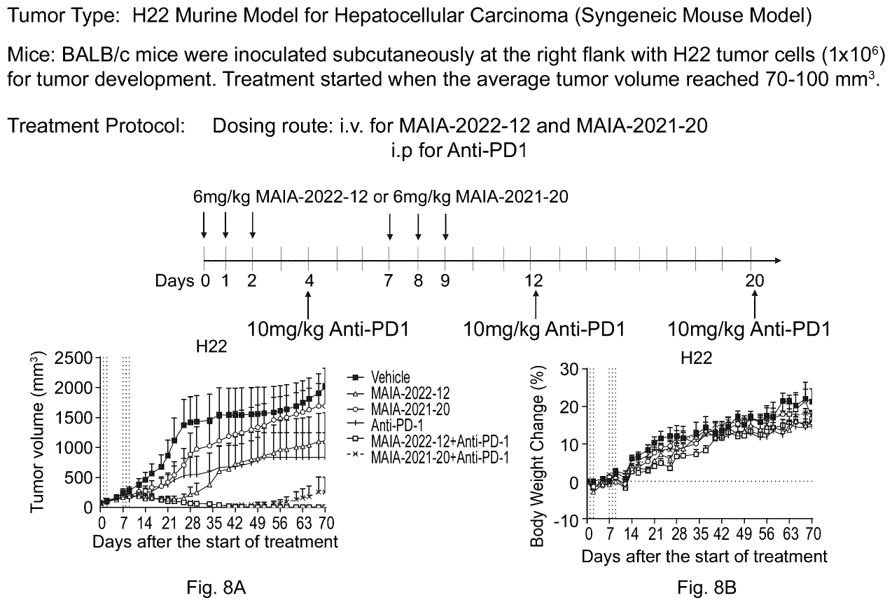

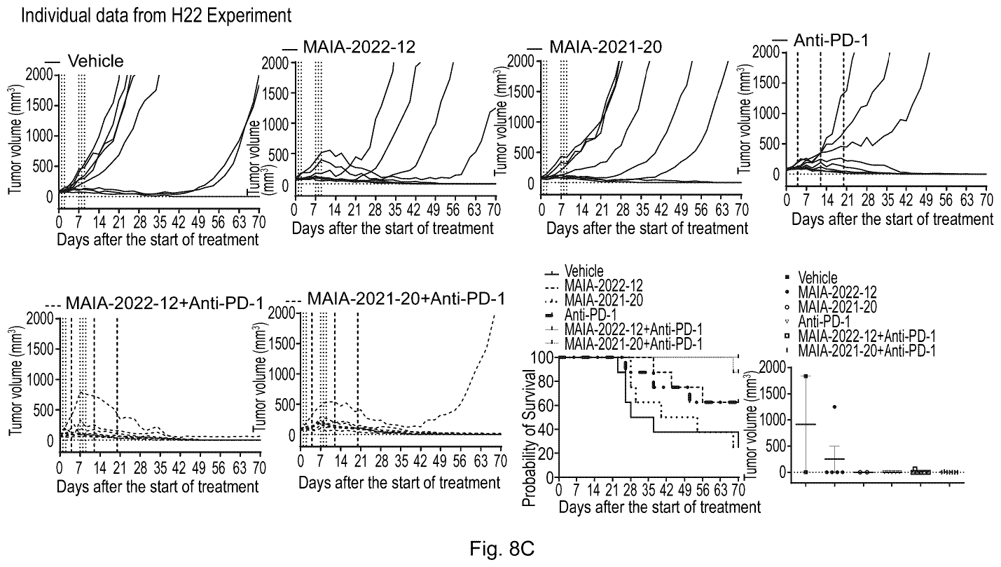

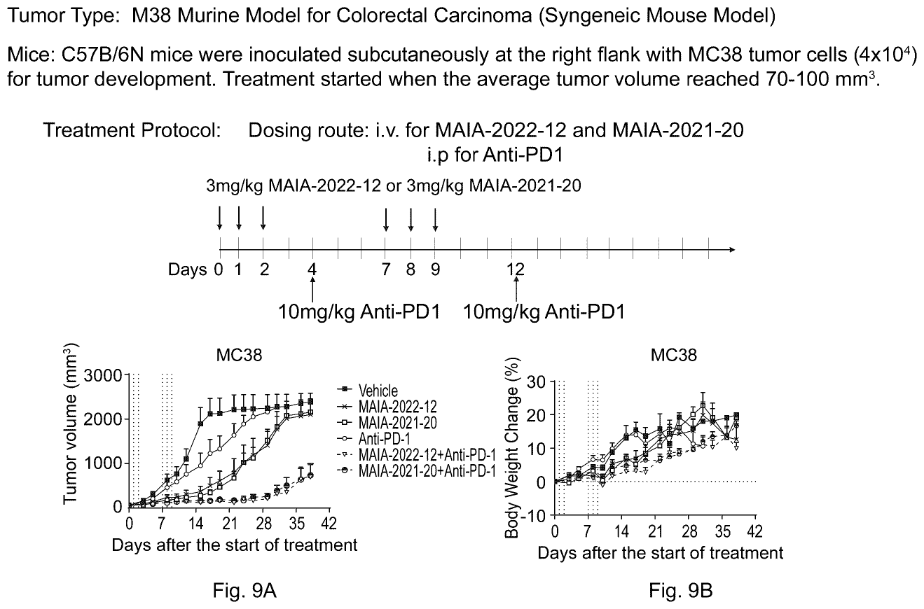

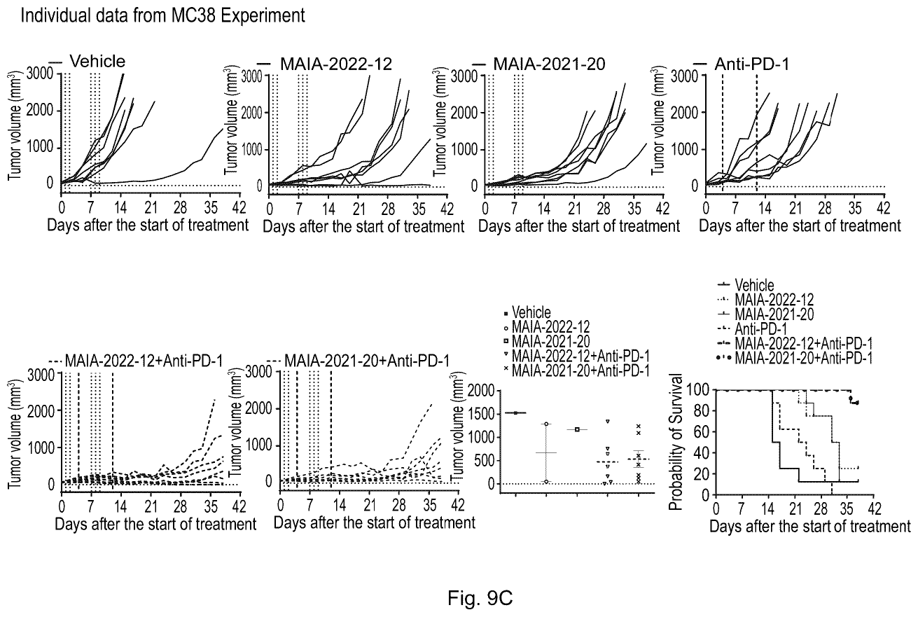

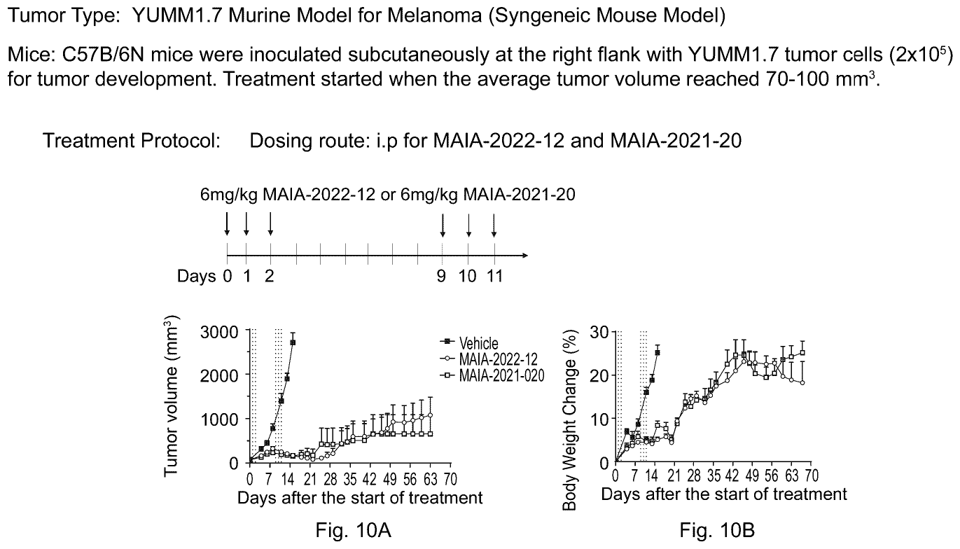

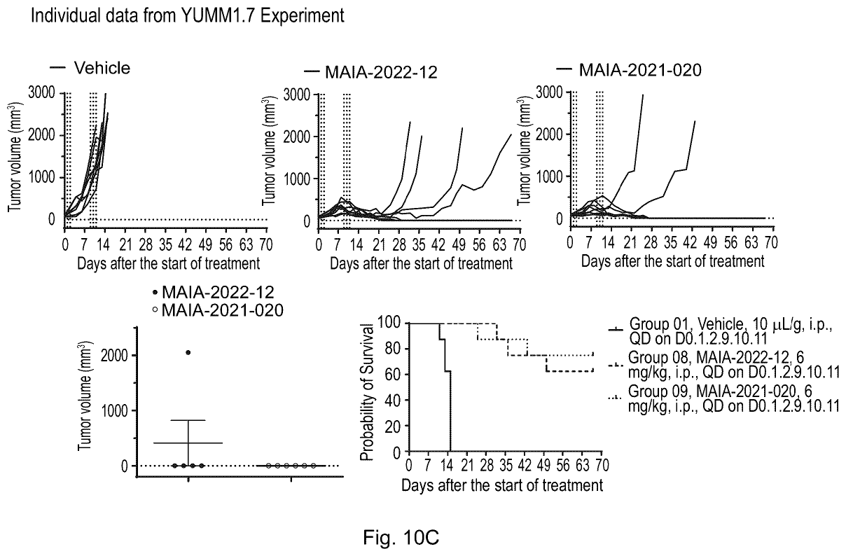

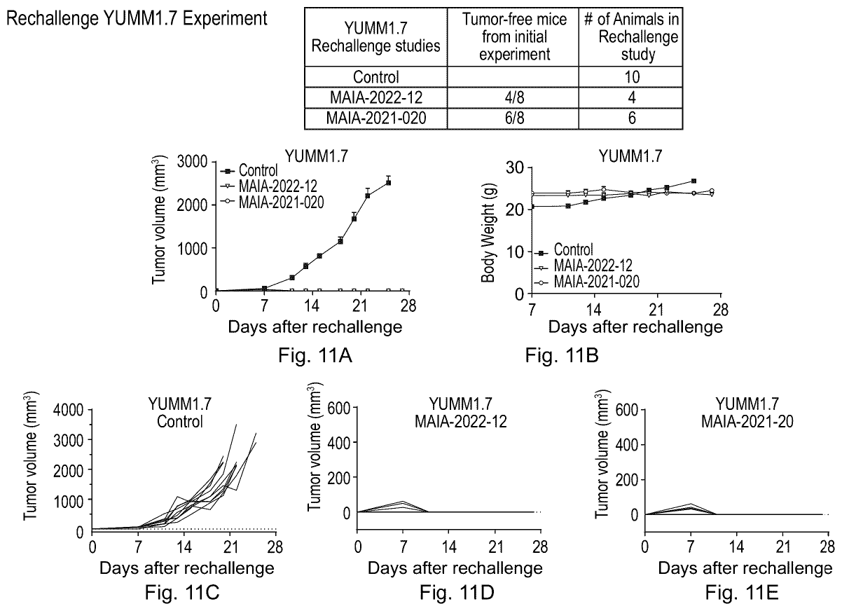

Representative dose-dependent anticancer activity data of some of the dinucleotide compounds disclosed herein are shown in the FIGS. 6 and 7.

Table 1 shows the IC₅₀ relative to untreated cells for compounds 1, 5, 6, 11 and 12 as well as enantiomers of compound 11 (Sp and Rp) and compound 12 (Sp and Rp) tested in human cell lines HEK293 (Kidney), Hep3B (liver; childhood HCC), HepG2 (liver; hepatoblastoma), NCI-H23 (lung; adenocarcinoma); H2081 (lung; SCLC).

TABLE 1
determination of IC₅₀ in different human cancer cell lines for various dinucleotides disclosed herein.

| | HEK293 | | Hep3B | | HepG2 | | NCI-H23 | | Panc-1 | |
|---|---|---|---|---|---|---|---|---|---|---|
| | IC₅₀ [μM] | % max. | IC₅₀ [μM] | % max. | IC₅₀ [μM] | % max. | IC₅₀ [μM] | % max. | IC₅₀ [μM] | % max. |
| | relative to | toxicity | relative to | toxicity | relative to | toxicity | relative to | toxicity | relative to | toxicity |
| | untreated | @ 50 | untreated | @ 50 | untreated | @ 50 | untreated | @ 50 | untreated | @ 50 |
| Dinucleotide ID | cells | μM | cells | μM2 | cells | μM3 | cells | μM4 | cells | μM5 |
| Compound 1 | 0.28 | 94 | 0.68 | 92 | 0.15 | 94 | 0.07 | 93 | 0.18 | 95 |
| Compound 5 | 0.02 | 94 | 0.09 | 87 | 0.04 | 92 | 0.01 | 94 | 0.25 | 96 |
| Compound 6 | 0.01 | 93 | 0.06 | 82 | 0.06 | 89 | 0.02 | 92 | 0.25 | 88 |
| Compound 11 | 0.63 | 86 | 1.12 | 62 | 0.61 | 71 | 1.11 | 79 | 5.12 | 80 |
| Compound 11 | 0.20 | 88 | 0.42 | 73 | 0.60 | 75 | 0.77 | 83 | 2.54 | 84 |
| enantiomer-Rp | | | | | | | | | | |
| Compound 11 | 6.07 | 85 | 36.55 | 56 | >50 | 47 | 12.83 | 76 | >50 | 26 |
| enantiomer -Sp | | | | | | | | | | |
| Compound-12 | 0.61 | 86 | 3.68 | 59 | 1.15 | 49 | 2.55 | 81 | >50 | 48 |
| Compound 12- | 0.43 | 86 | 2.83 | 74 | >50 | 46 | 2.04 | 84 | >50 | 52 |
| enantiomer Rp | | | | | | | | | | |
| Compound 12- | 3.43 | 86 | 19.69 | 62 | >50 | 48 | 16.18 | 74 | 16.18 | 74 |
| anantiomerSp | | | | | | | | | | |

cells were seeded at a density of 10.000 cells per well on 96 well plates, treated with the dinucleotides at the indicated doses and incubated for 96 h before analysis.

FIG. 6 shows the anticancer activity of some of the disclosed dinucleotides and representative dose response curves from which the IC₅₀ values reported in Table 1 were determined.

Specificity of the tested dinucleotides toward telomerase-positive cancer cell vs. telomerase-negative normal non-cancerous cell was demonstrated by testing these molecules in normal human BJ fibroblasts—FIG. 7. Importantly, these data show that in normal cells, the tested dinucleotides were not noticeably cytotoxic at the concentrations more than 100-fold higher, than the observed EC₅₀ values in cancer cells, demonstrating specificity towards telomerase-positive cancer cells.

The dinucleotide compounds were synthesized using solid phase-supported phosphoramidite method, and appropriately sugar ring- and base-protected nucleoside 3′-phosphoramidite building blocks (all commercially available from Glen Research). The products were cleaved from the universal solid support (Glen Research) and fully deprotected with aqueous ammonia, partially concentrated in vacuo, (to remove ammonia), and then purified by RP HPLC. The molecular weights and purity of the final dinucleotide products were confirmed by LC MS.

### Pharmaceutical Formulations and Routes of Administration

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render drugs stable and allow for uptake by target cells. Aqueous compositions of the present disclosure comprise an effective amount of the drug dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the agents of the compositions.

The active compositions of the present disclosure may include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or intratumoral or regional to a tumor, such as in the tumor vasculature. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present disclosure generally may be formulated in a neutral or salt form. Pharmaceutically acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The actual dosage amount of a composition in accordance with certain embodiments of the present disclosure administered to subject can be determined by physical and physiological factors such as the specific compound employed, the age, general health of the subject, diet, body weight, severity of condition, the type of disease being treated, previous or concurrent therapeutic interventions, idiopathy of the patient, absorption rates, distribution rates, inactivation rates, excretion rates, time of administration, the route of administration, and on the, judgment of the person supervising the administration. Depending upon the dosage and the route of administration, the number of administrations of a preferred dosage, and/or an effective amount may vary according to the response of the subject. The active ingredient may be administered at once, or may be divided into a number of smaller doses to be administered at varying intervals of time. As such, it is understood that for any particular subject, specific dosage regimens could be adjusted over time according to the individual need and the professional judgment of the person administering or supervising the administration of the compositions, and that the concentration ranges set forth herein are exemplary only and are not intended to limit the scope or practice of the claimed composition.

The dinucleotide compounds disclosed herein are included in the pharmaceutically acceptable carrier or diluent in an amount sufficient to deliver to a patient a therapeutically effective amount for the desired indication, without causing serious toxic effects in the patient treated. A preferred dose of the dinucleotide for all of the herein-mentioned conditions is in the range from about 10 ng/kg to 100 mg/kg, preferably 0.1 to 50 mg/kg per day, more generally 0.5 to about 25 mg per kilogram body weight of the subject per day. By way of non-limiting example, a typical dosage can range from 50.01-20% wt/wt in a suitable carrier. Similarly, the compound can be administered in any suitable unit dosage form, including, but not limited to, one containing less that 1 mg, 1 mg to 3000 mg, or 5 to 1000 mg of dinucleotide compound disclosed herein.

Compositions may be administered on an ongoing or continuous basis; on an as needed basis; or 1, 2, 3, 4, 5, 6, 5, 8, 9, 10, 11, 12, 13, 14, 15, 16, 17, 18, 19, 20 or more times including but not limited to one containing less than 1 mg.

### Combined Therapy

In order to increase the effectiveness a treatment, it may be desirable to combine compositions of the present disclosure with a second treatment or pharmaceutical composition. For example, a method of use can further include administration of a second pharmaceutical composition comprising an anti-cancer agent or other agent effective in the treatment of hyperproliferative disease. An anti-cancer agent can negatively affect cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, a second pharmaceutical composition can be administered in an effective amount or combined effective amount to kill or inhibit proliferation of certain cells.

In some embodiments, a method of treatment can comprise a simultaneous co-administration. This process may involve administration at the same time or sequentially. Co-administration can be achieved by contacting the cell with a single composition or pharmaceutical formulation that includes one or more of the dinucleotide compounds disclosed herein and another anti-cancer agent, or by contacting the cell with two distinct compositions or formulations, at the same time, wherein one composition includes one or more of the dinucleotide compounds disclosed herein and the other includes the second agent(s). Similarly, two compositions can be administered not at the same time but in temporal proximity to each other, e.g., on the same day or within the same week.

In other embodiments a method of treatment can comprise a first stage wherein a pharmaceutical composition comprising one or more of the dinucleotide compounds disclosed herein is administered and a second stage where a second pharmaceutical composition is administered. The first stage and the second stage may be sequential in time, spaced apart in time (minutes, days, weeks, or months), or overlapping in time. In addition, the sequential order of treatment stages can be reversed or repeated.

To be sure, any combination of treatment stages may be employed. By way of example, administration one or more of the dinucleotide compounds disclosed herein is “A” and the treatment with a secondary agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In the context of the present disclosure, it is contemplated that administration of a pharmaceutical composition comprising a one or more of the dinucleotide compounds disclosed herein could be used in conjunction with a treatment B, such as gene therapy, chemotherapeutic, radiotherapeutic, or immunotherapeutic intervention, in addition to other pro-apoptotic or cell cycle regulating agents. It also is contemplated that various standard therapies, as well as surgical intervention, may be applied in combination with the described telomere shortening and telomere dysfunction-inducing therapy.

a. Chemotherapy

Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, farnesyl-protein transferase inhibitors, transplatinum, 5-fluorouracil, vincristin, vinblastin and methotrexate, or any analog or derivative variant of the foregoing.

b. Radiotherapy

Radiotherapies can cause DNA damage and include what are commonly known as X-rays, and/or the directed delivery of radioisotopes to tumor cells. Other forms of DNA damaging factors are also contemplated such as microwaves and UV-irradiation.

c. Immunotherapy

Immunotherapeutics, generally, rely on the use of immune effector cells and molecules to target and destroy cancer cells. Immunotherapy, thus, could be used as part of a combined therapy, in conjunction with the administration of a pharmaceutical composition comprising a one or more of the dinucleotide compounds disclosed herein. Immunotherapy modality relates to the targeting of the tumor cell through some marker of the tumor cell that is amenable to targeting, i.e., is not present on the majority of other cells. Many tumor markers exist and any of these may be suitable for targeting with a second treatment modality in the context of the present disclosure. Common tumor markers include carcinoembryonic antigen, prostate specific antigen, urinary tumor associated antigen, fetal antigen, tyrosinase (p9'7), gp68, TAG-72, HMFG, Sialyl Lewis Antigen, MucA, MucB, PLAP, estrogen receptor, laminin receptor, erb B and p155

d. Surgery

Curative surgery is a cancer treatment that can be used in conjunction with a pharmaceutical composition comprising a one or more of the dinucleotide compounds disclosed herein. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). It is further contemplated that composition of the present disclosure can be administered in conjunction with removal of superficial cancers, precancers, or incidental amounts of normal tissue. Upon excision of part of all of cancerous cells, tissue, or tumor, a cavity may be formed in the body. Treatment may be accomplished by administration of a pharmaceutical composition comprising a one or more of the dinucleotide compounds disclosed herein.

e. Other Anti-Cancer Agents

It is contemplated that other anti-cancer agents may be used in combination with comprising a one or more of the dinucleotide compounds disclosed herein to additively or synergistically enhance the therapeutic efficacy of treatment.

These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR₄ or DR_{5/}TRAIL would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other anti-cancer agents that increase the sensitivity of a hyperproliferative cell to apoptosis, and signal transduction inhibitors like the antibody c225, could be used in combination one or more of the dinucleotide compounds disclosed herein to improve the treatment efficacy.

Lastly, additional agents can also include anti-cancer agents which are broadly characterized as anti-metabolites, inhibitors of topoisomerase I and II, alkylating agents and microtubule inhibitors (e.g., taxol). Anti-cancer agents for use in the present invention include, for example, Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); mechlorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others.

Hormonal therapy may also be used in combination with the administration of a pharmaceutical composition may be used in combination with one or more of the dinucleotide compounds disclosed herein. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

The present disclosure provides for sequential treatment of cancers using the dinucleotide compounds disclosed herein followed by PD-L1, PD-1 and/or CTLA-4 therapy. The periods for each treatment may vary and it is contemplated that short gap between treatments will be advantageous. For example, for the dinucleotide compounds disclosed herein the treatment may be as little as 2 days but may be 3, 4 or more days, including 2-4 days. The gap prior to PD-L1, PD-1 and/or CTLA-4 treatment should be at least one day and may be up 14 days, such as 2-4 days. An overlap between the dinucleotide compounds disclosed herein and PD-L1, PD-1 and/or CTLA-4 might need to be avoided due to potentially detrimental effects of the dinucleotide compounds disclosed herein on activated effector T cells.

The daily dosage of dinucleotide compounds disclosed herein will be between 0.5 mg/kg and 10 mg/kg, preferably intravenous or oral. The dose of PD-L1, PD-1 and/or CTLA-4 will be consistent with approved current dosing schedules.

In the context of the present disclosure, it also is contemplated that the dinucleotide compounds disclosed herein and anti PD-L1 such as atezolizumab or a dinucleotide and anti PD-1 such as Libtayo® or anti CTAL-4 could be used in conjunction with chemo- or radiotherapeutic intervention, or other treatments. It also may prove effective, in particular, to combine a dinucleotide compound as disclosed herein, anti PD-L1, anti PD-1 or anti CTLA-4 with other therapies that target different aspects of cancer cell function.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with a dinucleotide compound disclosed herein and at least one other agent. These compositions would be provided in a sequential or combined amount effective to kill or inhibit proliferation of the cell. This process may involve contacting the cells with dinucleotide/anti PD-L1, anti PD-1 or anti CTLA-4 and the other agent(s) or factor(s) at the same time. This may be achieved by contacting the cell with a single composition or pharmacological formulation that includes both agents, or by contacting the cell with two distinct compositions or formulations, at the same time, wherein one composition includes the interferon prodrugs according to the present disclosure and the other includes the other agent.

Alternatively, the dinucleotid/anti PD-L1, anti PD-1 or anti CTLA-4 therapy may precede or follow the other agent treatment by intervals ranging from minutes to weeks. In embodiments where the other agent and the interferon prodrugs are applied separately to the cell, one would generally ensure that a significant period of time did not expire between each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either interferon prodrugs or the other agent will be desired. Various combinations may be employed, where a dinucleotide compound disclosed herein/anti PD-L1, anti PD-1 or anti CTLA-4 therapy is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve cell killing, both agents are delivered to a cell in a combined amount effective to kill the cell.

Agents or factors suitable for cancer therapy include any chemical compound or treatment method that induces DNA damage when applied to a cell. Such agents and factors include radiation and waves that induce DNA damage such as, irradiation, microwaves, electronic emissions, and the like. A variety of chemical compounds, also described as “chemotherapeutic” or “genotoxic agents,” may be used. This may be achieved by irradiating the localized tumor site; alternatively, the tumor cells may be contacted with the agent by administering to the subject a therapeutically effective amount of a pharmaceutical composition.

Various classes of chemotherapeutic agents are contemplated for use with the present disclosure. Other chemotherapeutics include selective estrogen receptor antagonists (“SERMs”), such as Tamoxifen, 4-hydroxy Tamoxifen (Afimoxfene), Falsodex, Raloxifene, Bazedoxifene, Clomifene, Femarelle, Lasofoxifene, Ormeloxifene, and Toremifene. The agents camptothecin, actinomycin-D, and mitomycin C are commonly used chemotherapeutic drugs. The disclosure also encompasses the use of a combination of one or more DNA damaging agents, whether radiation-based or actual compounds, such as the use of X-rays with cisplatin or the use of cisplatin with etoposide. The agent may be prepared and used as a combined therapeutic composition.

Heat shock protein 90 is a regulatory protein found in many eukaryotic cells. HSP90 inhibitors have been shown to be useful in the treatment of cancer. Such inhibitors include Geldanamycin, 17-(Allylamino)-17-demethoxygeldanamycin, PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21-day intervals for doxorubicin, to 35-50 mg/m² for etoposide intravenously or double the intravenous dose orally. Microtubule inhibitors, such as taxanes, also are contemplated. These molecules are diterpenes produced by the plants of the genus Taxus and include paclitaxel and docetaxel.

Epidermal growth factor receptor inhibitors, such as Iressa, mTOR, the mammalian target of rapamycin (also known as FK506-binding protein 12-rapamycin associated protein 1 (FRAP1)), is a serine/threonine protein kinase that regulates cell growth, cell proliferation, cell motility, cell survival, protein synthesis, and transcription. Rapamycin and analogs thereof (“rapalogs”) are therefore contemplated for use in cancer therapy in accordance with the present disclosure. Another EGFR inhibitor of particular utility here is Gefitinib.

Another possible therapy is TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenesis and viral replication.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as y-rays, x-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In addition, it also is contemplated that a distinct immunotherapy, a hormone therapy, a toxin therapy and/or surgery can be used.

The skilled artisan is directed to “Remington' s Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Experimental:

TABLE 2
List of Abbreviations

| | |
|---|---|
| ACN | Acetonitrile |
| CAS | Chemical Abstracts Service |
| conc. | concentration |
| Da | Dalton |
| g | gram |
| h | hours |
| HPLC | High Performance Liquid |
| | Chromatography |
| M | molar |
| min | minutes |
| mM | millimolar |
| M.W. | Molecular weight |
| Na+ | Sodium ion |
| QC | Quality Control |
| RT | Room temperature |
| TEA+ | Triethylammonium ion |
| TEAA | Triethylammonium acetate |
| UV | Ultra-violet |
| w.r.t. | With respect to |

### Process Summary:

The manufacturing of the disclosed dinucleotides involved the following process steps:
1. Solid-phase synthesis on a H8-custom solid-phase synthesizer; using universal-linker—CPG as solid support.
2. Cleavage and deprotection.
3. Purification by reversed-phase (RP) HPLC (column: Waters Xbridge C18 (19 mm×50 mm, 14.2 mL); Eluent A: 100 mM TEAA in Water, Eluent B: 100 mM TEAA in 25% ACN-water as eluent system).
4. If deemed necessary: Buffer exchange was performed by RP-HPLC (Column: Waters Xbridge C18 (19 mm×50 mm, 14.2 mL); Eluent A: Water, Eluent B: ACN, as eluent system).
5. Lyophilization cycles and then final weight measurement after 2 cycles (amount finalization).

### Materials and General Procedures:

### Building Blocks and Solid Supports

The phosphoramidite building blocks and CPG-solid supports used in the solid phase synthesis are summarized below in Table 3.

TABLE 3
Building blocks and solid supports used in solid phase synthesis.

| Chemical name/CAS# | Chemical structure | Supplier/M.W. |
|---|---|---|
| 6-thio-dG-CE phosphoramidite 5′-Dimethoxytrityl-N₂-trifluoroacetyl-2′-deoxy-6-(2- cyanoethyl)thio-Guanosine,3′-[(2- cyanoethyl)-(N,N-diisopropyl)]-phosphoramidite CAS# 146691-59-0 | 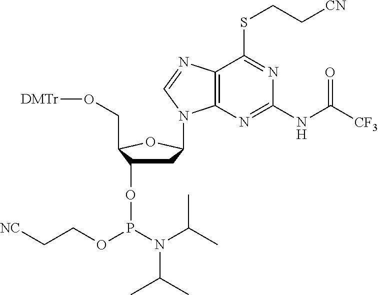 | Glen Research # 10-1072-02M C₄₅H₅₀F₃N₈O₇PS (M.W. 934.97) |
| 6-Thio-G-CE phosphoramidite 5′-Dimethoxytrityl-N₂-isobutyryl-6-(2-cyanoethyl)thio- Guanosine,2′-O-TBDMS-3′-[(2-cyanoethyl)-(N,N- diisopropyl)]-phosphoramidite CAS# not assigned | 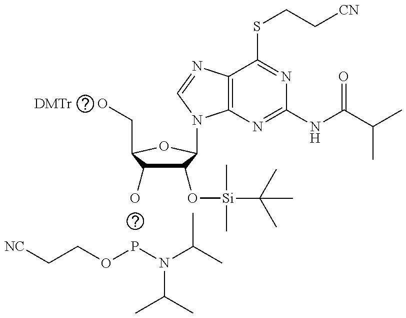 | Glen Research # 10-3072-02M C₅₃H₇₁N₈O₈PSSi (M.W. 1038.32) |
| 5-F-dU-CE phosphoramidite 5′-Dimethoxytrityl-5-fluoro-2′-dioxyUridine,3′-[(2- cyanoethyl)-(N,N-diisopropyl)]-phosphoramidite CAS# 142246-63-7 | 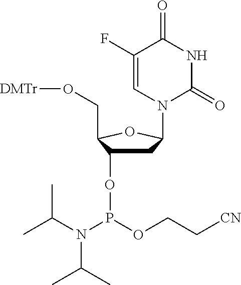 | Glen Research # 10-1092-02M C₃₉H₄₆FN₄O₈P (M.W. 748.79) |
| 4T-dT-CE phosphoramidite 5′-Dimethoxytrityl-2′-deoxy-4-(2-cyanoethylthio)- Thymidine,3′-[(2-cyanoethyl)-(N,N-diisopropyl)]- phosphoramidite CAS# 1290537-73-3 | 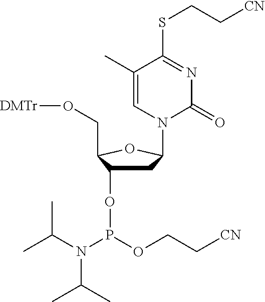 | Glen Research # 10-1034-02M C₄₃H₅₂N₅O₇PS (M.W. 813.95) |
| 5′-Palmitate-C6-CE phosphoramidite 2-Cyanoethyl-(6-palmitalmidohexyl)- diisopropylphosphoramidite CAS# not assigned | 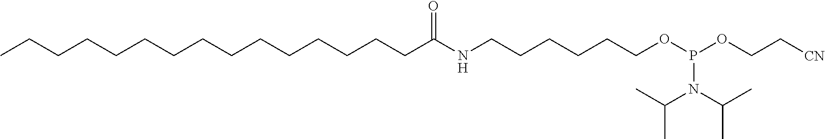 [SMILES: CCCCCCCCCCCCCCCC(=O)NCCCCCCOP(OCCC#N)N(C(C)C)C(C)C] | Link Technoloties # 2199 C₃₁H₆₂FN₃O₃P (M.W. 555.45) |
| Phosphate-ON reagent 2-[2-(4,4′- Dimethoxytrityloxy)ethylsulfonyl]ethyl-(2- cyanoethyl)-(N,N-diisopropyl)-phosphoramidite CAS# 108783-02-4 | 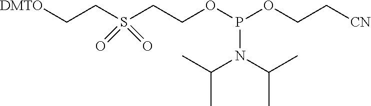 | Chemgenes # CLP-1544 C34H45N2O7PS (M.W. 656.8) |
| Solid support: Universal UnyLinker Support 500Å CPG Loading: 84.8 μmol/g | 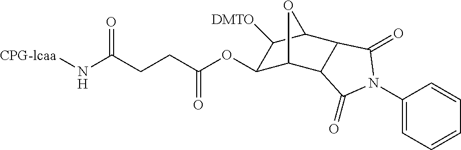 | ChemGenes Corp. # N-4000-05 |

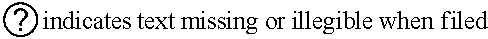

### Solvents and Ancillary Reagents:

All Solvents and ancillary reagents used in these projects are summarized in Table 4.

TABLE 4
Solvents, reagents and auxiliary materials used in solid
phase synthesis and cleavage/deprotection step.

| Material | Formulation | Supplier | Product# |
|---|---|---|---|
| Acetonitrile (ACN) | ≤30 ppm water | Sigma- | L010000 |
| | | Aldrich | |
| Activator | 5-Ethylthio-1H-tetrazole (ETT), 0.5M | Biosolve | 22112402 |
| | in ACN | | |
| Deblock | Dichloroacetic acid (3%) in toluene | Sigma- | L023050 |
| | (v/v) | Aldrich | |
| Cap A | Acetic anhydride/tetrahydrofuran | Sigma | L040080 |
| | 9.1:90.9 (v/v) | Aldrich | |
| Cap B | Tetrahydrofuran/N- | Sigma | L050080 |
| | methylimidazole/pyridine 8:1:1 (v/v/v) | Aldrich | |
| Oxidizer | 700 mM tBuOOH (5.5M in decane) | Sigma | 19997 |
| | in ACN-Toluene (1.1:6:1, v/v/v) | Aldrich | |
| Sulfurizer | 3-Amino-1,2,4-dithiazole-5-thione | TCI | X0001 |
| | (Purity: >96.0% (HPLC)) dissolved in | Chemicals | |
| | ACN-pyridine (2:3 v/v) (100 mM) | | |
| 1,8- | 1M DBU in ACN | Sigma | 508906 |
| Diazabicyclo[5.4.0]undec-7- | | Aldrich | |
| en (1,5-5) (DBU) | | | |
| Pyridine | Anhydrous, 99.8% | Sigma- | 270970 |
| | | Aldrich | |
| Ammonia | Solution (30%, puriss) in water | Sigma- | 221228 |
| | | Aldrich | |
| Methylamine | 41 wt. % in water | Fluka | 65580 |
| Triethylamine | 37% Hydrogenfluoride in | Sigma- | 344648 |
| trishydrofluoride (TEA•3HF) | triethylamine | Aldrich | |
| Molecular sieves | 3 Å, 8-12 mesh | Carl Roth | N893.1 |
| Argon | 5.0 | Rieβner | RH5 |
| | | Gase | |

### Solid Phase Synthesis of Dinucleotides:

Synthesis was performed according to the conventional solid-phase oligonucleotide synthesis protocol using standard phosphoramidite chemistry on an automated solid-phase synthesizer. An H8-Custom Synthesizer (K&A Laborgerate) controlled by the H8-COM software package was used to perform the solid phase synthesis.

Phosphoramidites were dissolved in dry acetonitrile to give a 0.1 M solution which were thereafter dried over molecular sieves (3 Å) for a minimum of two hours before being installed at predefined amidite-ports on the H8-synthesizer.

As activator, 0.5 M 5-Ethylthio-1H-tetrazole (ETT) in ACN was used and dried over molecular sieves (3 Å) for at least 24 h prior to synthesis initiation. As sulfurizing reagent 3-Amino-1,2,4-dithiazole-5-thione (or Xanthane hydride; 100 mM in ACN-pyridine (2:3 v/v)) was freshly formulated prior to use.

All other ancillary reagents for the solid phase synthesis were used as received from the respective commercial sources (Table 5).

During the synthetic cycle, a standard oxidation-step followed by capping-step was employed. All dinucleotides were synthesized in DMT-OFF mode. Syntheses involving Phosphate-ON reagent were performed omitting the final capping step. A detailed list of all raw materials, ancillary reagents and solvents with their suppliers and product numbers is listed in Table 3 and Table 4.

### Cleavage and Deprotection:

After completion of the synthesis, dinucleotides still immobilized onto the solid supports were treated with DBU at ambient temperature for four hours, to ensure the removal of the cyanoethyl protecting group, especially from the 6-thio position of the 6SdG or 6SG and 4-thio position of the 4TdT nucleotide units. Thereafter the CPG-solid supports bearing products were subsequently dried at room temperature using a vacuum pump and the resultant dry CPG in each case was transferred into a 15 mL-Falcon tube. To each tube AMA (methylamine in water (41% wt.)+aq. NH3 (30%, puriss) were pre-mixed 1:1 v/v) solution was added (3 mL) and the tubes were shaken at 30° C. for three hours. This effected the cleavage of dinucleotide from the solid support, de-phosphorylation of unilinker at the 3′-end to yield free 3′-OH and deprotection of the 2-NH2 positions of 6SdG and 6SG by removing trifluoroacetyl-and isobutyryl-protecting groups. In case of dinucleotides comprising 6SG, 2′-O-TBDMS deprotection was performed thereafter by adding TEA·3HF to the deprotection mixture, and then the mixture was shaken for additional 90 minutes at 45° C. Finally, the dinucleotides dissolved in the supernatant solutions were separated from the solid supports by centrifugation (using a HERAEUS Multifuge X3F by Thermo Scientific) followed by decantation. The residual CPG in each tube was washed with water to collect any remaining solution containing dinucleotide. The combined liquid phases were concentrated in vacuo. The residue was diluted with a TEAA buffer solution (100 mM TEAA in water) for subsequent HPLC purification.

### Purification by RP-HPLC:

Purification was performed on an Akta pure 150 HPLC system using an RP Xbridge C18 column from Waters. The details of the process are listed below and Table 5 summarizes the materials that were used for the purification and the subsequent desalting step.

### Äkta Pure 150 HPLC System

### Reversed-Phase Chromatography:

Column—X-Bridge C18 (19 mm×50 mm, 14.2 mL);
Eluent A: 100 mM TEAA in water
Eluent B: 100 mM TEAA in an ACN-water mixture (25% ACN in water)

TABLE 5
Solvents, reagents and auxiliary materials used
for RP-HPLC purification and desalting.

| | Quality/ | | |
|---|---|---|---|
| Material | Formulation | Supplier | Product# |
| XBridge Prep C18 | Bead diameter: | Waters | 186002977 |
| OBD | 5 μm | | |
| Sodium chloride | 5M; BioReagent | Sigma | S5150 |
| Triethylammonium | 1M; pH 7.0 | PanReac | A3846 |
| acetate | | AppliChem | |
| Acetonitrile | for DNA Synthesis | Sigma-Aldrich | L010000 |

### Desalting:

In case of samples meant for in vivo studies, the counter cation TEA⁺ were replaced by Na⁺ from the purified Material by performing a buffer exchange. After removal of RP-HPLC eluent buffers by evaporation and subsequent lyophilization, the purified material was reconstituted in 500 mM NaCl solution and subjected to buffer exchange by RP-HPLC on an Äkta pure 150 using a Waters X-Bridge C18 column (19 mm×50 mm, 14.2 mL). The details are listed below and the materials used are summarized in Table 5.

### Äkta Pure 150

### Reversed-Phase Chromatography:

Column—X-Bridge C18 (19×50 mm; 14.2 mL);
Eluent A: water
Eluent B: 95% ACN-water

Thereafter the acetonitrile-water was removed in vacuo and the aqueous dinucleotide solution was lyophilized.

### Lyophilization and Quantification:

The oligonucleotide solution was distributed into Falcon tubes and lyophilized (using a CHRIST Epsilon 1-4 LSCplus lyophilizer). After first lyo-cycle, the residual dinucleotide was re-dissolved in water and lyophilized again. This procedure was repeated until a constant weight was achieved. Prior to the final freeze-dying step, sufficient amounts for quality control testing were aliquoted. After completion of the freeze drying step, the dry material was quantified by weighing.

### QC-Analysis:

For QC release testing UV-based RP-chromatogram traces of the dinucleotides were recorded at both 260 nm and 350 nm (characteristic Absorption maxima of the 6SG-nucleobase; N. F. Krynetskaia, X. Cai, J. L. Nitiss, E. Y. Krynetski, M. V, Relling, FASEB J. 2000, 14, 2339). While both values were reported, traces at 350 nm were monitored and integrated for final purity determination, as agreed with sponsor. In order to confirm purity and identity of dinucleotides were performed on an Ultimate 3000 HPLC system (Thermo Scientific) equipped with an Acquity UPLC Oligonucleotide C18 column from Waters (2.1 mm×100 mm, 0.37 mL).

Data acquisition and integration of HPLC chromatograms were performed using the software package Chromeleon from Thermo Fisher Scientific. For every sample the peaks were integrated by vertical peak splitting. In case of overlapping peaks, split was made at minima between the two and their identity was corroborated from the associated MS-Spectra.

ESI/MS detection was used to analyze the monoisotopic molecular weight of the samples. For this purpose, an Ultimate 3000 HPLC system equipped with an UV detector as well as a qTOF detector (Compact) from Broker Daltonics was used. The MS spectra were extracted in the Total Ion Current (TIC) using the Broker Hystar Data Analysis software. Identity of all the dinucleotides were also established within the agreed specification.

### Syngeneic Mouse Models:

### A) Tumor Type: H22 Murine Model for Hepatocellular Carcinoma (Syngeneic Mouse Model

Method: Female BALB/c mice were inoculated subcutaneously at the right flank with H22 tumor cells (1×106) to establish a syngeneic subcutaneous H22 tumor model. When the average tumor volume reached 70-100 mm3, mice were assigned to 6 groups randomly based on tumor volume and body weight: the control group (vehicle, i.v., QD on D0, D1, D2, D7, D8, D9), the MAIA-2022-12 group (MAIA-2022-12, i.v., 6 mg/kg, QD on D0, D1, D2, D7, D8, D9), the MAIA-2021-20 group (MAIA-2021-20, i.v., 6 mg/kg, QD on D0, D1, D2, D7, D8, D9), the Anti-PD-1 group (Anti-PD-1, i.p., 10 mg/kg, QD on D4, D12 and D20), the MAIA-2022-12 combination group (MAIA-2022-12, i.v., 6 mg/kg, QD on D0, D1, D2, D7, D8, D9; Anti-PD-1, i.p., 10 mg/kg, QD on D4, D12 and D20), the MAIA-2021-20 combination group (MAIA-2021-20, i.v., 6 mg/kg, QD on D0, D1, D2, D7, D8, D9; Anti-PD-1, i.p., 10 mg/kg, QD on D4, D12 and D20), with 8 mice per group. Dose volumes were 10 μL/g. Tumor volume calculations and survival analysis were used to evaluate drug efficacy, and body weight change and the animal death were used to evaluate drug safety.

### B) Tumor Type: M38 Murine Model for Colorectal Carcinoma (Syngeneic Mouse Model)

Method: Female C57BL/6N mice were inoculated subcutaneously at the right flank with MC38 tumor cells (4×104) to establish a syngeneic subcutaneous MC38 tumor model. When the average tumor volume reached 70-100 mm3, mice were assigned to 6 groups randomly based on tumor volume and body weight: the control group (vehicle, i.v., QD on D0, D1, D2, D7, D8, D9), the MAIA-2022-12 group (MAIA-2022-12, i.v., 3 mg/kg, QD on D0, D1, D2, D7, D8, D9), the MAIA-2021-20 group (MAIA-2021-20, i.v., 3 mg/kg, QD on D0, D1, D2, D7, D8, D9), the Anti-PD-1 group (Anti-PD-1, i.p., 10 mg/kg, QD on D4, D12), the MAIA-2022-12 combination group (MAIA-2022-12, i.v., 3 mg/kg, QD on D0, D1, D2, D7, D8, D9; Anti-PD-1, i.p., 10 mg/kg, QD on D4, D12), the MAIA-2021-20 combination group (MAIA-2021-20, i.v., 3 mg/kg, QD on D0, D1, D2, D7, D8, D9; Anti-PD-1, i.p., 10 mg/kg, QD on D4, D12), with 8 mice per group. Dose volumes were 10 μL/g. Tumor volume calculations and survival analysis were used to evaluate drug efficacy, and body weight change and the animal death were used to evaluate drug safety.

### c) Tumor Type: YUMM1.7 Murine Model for Melanoma (Syngeneic Mouse Model)

Method: Female C57BL/6N mice were inoculated subcutaneously at the right flank with YUMM1.7 tumor cells (2×105) to establish a syngeneic subcutaneous YUMM1.7 tumor model. When the average tumor volume reached 70-100 mm3, mice were assigned to 3 groups randomly based on tumor volume and body weight: the control group (vehicle, i.p., QD on D0, D1, D2, D9, D10, D11), the MAIA-2022-12 group (MAIA-2022-12, i.p., 6 mg/kg, QD on D0, D1, D2, D9, D10, D11), the MAIA-2021-20 group (MAIA-2021-20, i.p., 6 mg/kg, QD on D0, D1, D2, D9, D10, D11), with 8 mice per group. Dose volumes were 10 μL/g. Tumor volume calculations and survival analysis were used to evaluate drug efficacy, and body weight change and the animal death were used to evaluate drug safety.

### d) Rechallenge YUMM1.7 Experiment

Method: 4 mice in MAIA-2022-12 group and 6 mice MAIA-2021-020 group were tumor-free from the initial experiment (see slide 7 for the treatment schedule and the results). Rechallenge of YUMM1.7 was performed in these tumor-free mice to see if the mice gained immune protection effect after treatment.

Female C57BL/6N mice (10 mice for control group, 4 mice for MAIA-2022-12 and 6 mice for MAIA-2021-20) were inoculated subcutaneously at the left flank with YUMM1.7 tumor cells (2×105) 46 days after last treatment for tumor development. One week after tumor inoculation, mice were started to be measured for tumor development (2-3 times a week). Tumor volume calculations were used to see if mice formed tumor after inoculation and body weight change and the animal death were used to evaluate drug safety.

1. A compound comprising the structure of Formula I or a pharmaceutically acceptable salt thereof: 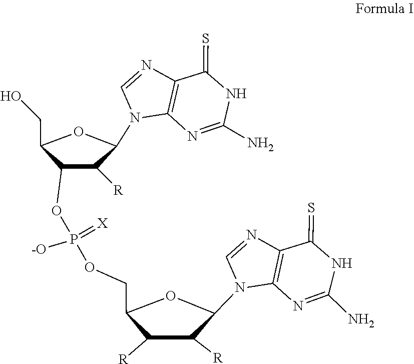
wherein R is independently H or OH and X is O or S.

2. A compound of claim 1, wherein X is O.

3. A compound of claim 2, wherein the compound has the following structure:

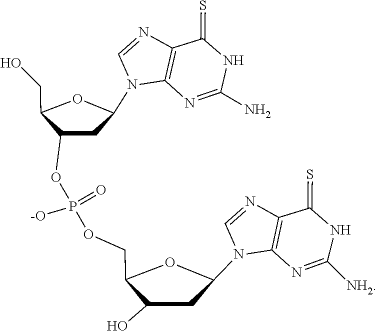

4-9. (canceled)

10. A compound or a pharmaceutically acceptable salt thereof having the following structure:

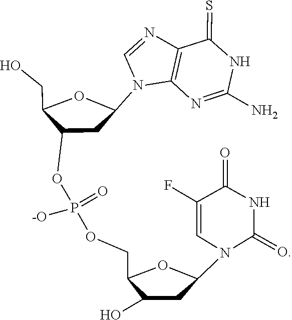

11. A compound or a pharmaceutically acceptable salt thereof having the following structure:

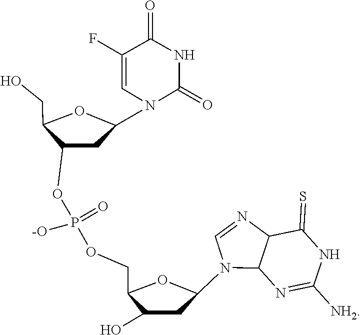

12-15. (canceled)

16. A compound comprising a structure of Formula II or a pharmaceutically acceptable salt thereof: 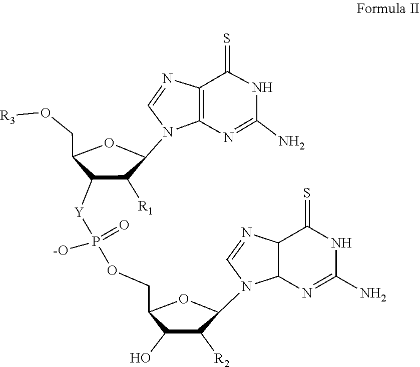
wherein,
R₁ and R₂ are independently H or OH;
X is O or S;
Y is O; and
R₃ is selected from the group consisting of a phosphate group, thio-phosphate group, palmitic acid, tocopherol, and cholesterol group.

17-32. (canceled)

33. A compound or a pharmaceutically acceptable salt thereof having the following structure:

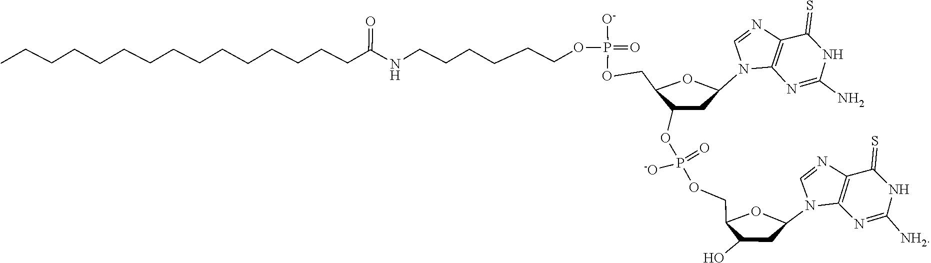

34-37. (canceled)

38. A compound or a pharmaceutically acceptable salt thereof having the following structure:

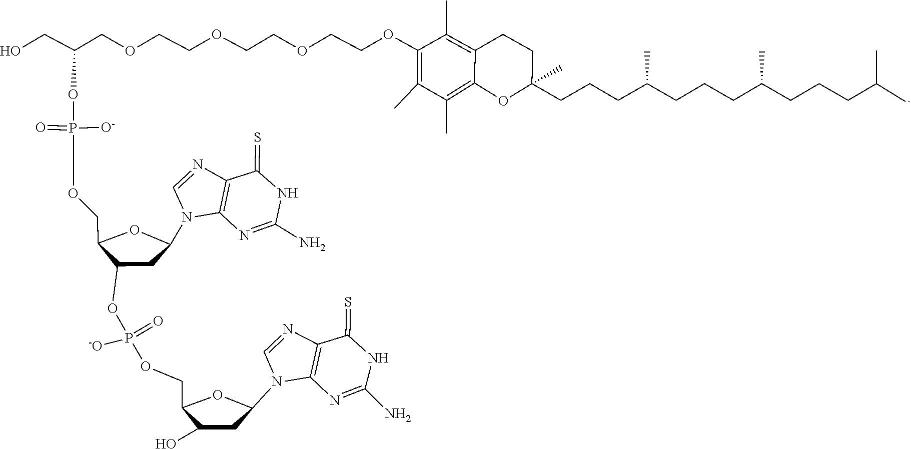

39-68. (canceled)

69. A compound comprising the structure of Formula III or a pharmaceutically acceptable salt thereof: 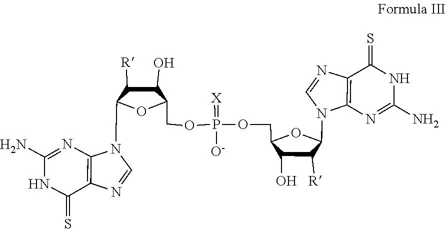
wherein X is O or S and R′ is independently H or OH.

70. A compound according to claim 69 having the following structure:

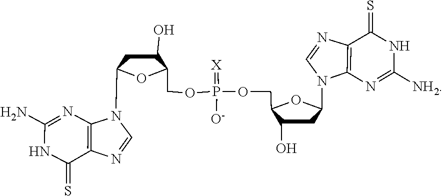

71-78. (canceled)

79. A compound or a pharmaceutically acceptable salt thereof having the following structure:

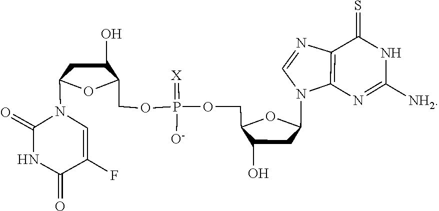

80-94. (canceled)

95. A compound comprising the structure of Formula VII or pharmaceutically acceptable salt thereof wherein X is O or S and R₁ and R₂ are H or OH.

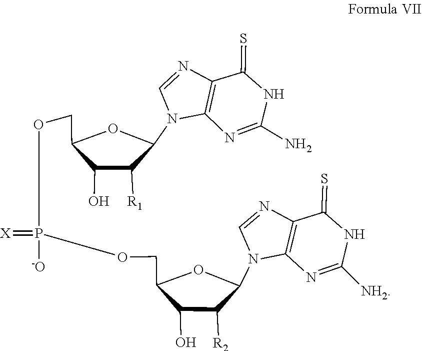

96-97. (canceled)

98. A compound according to claim 95 having the following structure:

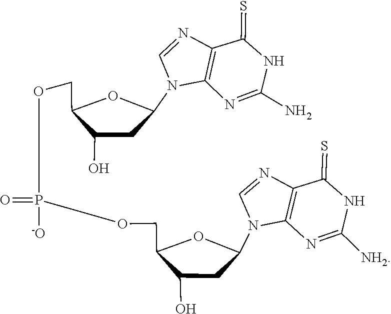

99-104. (canceled)

105-118. (canceled)
